(19) United States
(12) Patent Application Publication
(10) Pub. No.: US 2006/0110819 A1
(43) Pub. Date: May 25, 2006

(54) Apparatus and method for expression and capture of biomolecules and complexes on adsorbent surfaces

(76) Inventors: Lee O. Lomas, Pleasanton, CA (US); Egisto Boschetti, Croissy sur Seine (FR); Tai-Tung Yip, Cupertino, CA (US)

Correspondence Address: TOWNSEND AND TOWNSEND AND CREW LLP, TWO EMBARCADERO CENTER, 8TH FLOOR, SAN FRANCISCO, CA, 94111-3834, US

(21) Appl. No.: 10/528,820

(22) PCT Filed: Sep. 29, 2003
(86) PCT No.: PCT/US03/30760

§ 371 Date: May 16, 2005

Related U.S. Application Data
(60) Provisional application No. 60/415,224, filed on Sep. 30, 2002.
(60) Provisional application No. 60/420,172, filed on Oct. 21, 2002.

(51) Int. Cl. C12M 1/34

(52) U.S. Cl. 435/287.2

(57) ABSTRACT

The present invention relates to the fields of molecular biology, combinatorial chemistry and biochemistry. Particularly, the present invention describes apparatus and methods for the detection and isolation of binding partners and activity modulators for biomolecules. The apparatus described allows for expression, capture and analysis of one or more biomolecules in a single step.

### CROSS-REFERENCE TO RELATED APPLICATIONS

This application claims the benefit of U.S. Provisional Application No. 60/415,224 filed Sep. 30, 2002 and U.S. Provisional Application No. 60/420,172 filed Oct. 21, 2002, herein incorporated by reference.

### FIELD OF THE INVENTION

The present invention relates to the fields of molecular biology, combinatorial chemistry and biochemistry. Particularly, the present invention describes apparatus and methods for the expression, isolation and detection of binding partners and activity modulators for biomolecules. The apparatus described allows for expression, capture and analysis of one or more biomolecules in a single step.

### BACKGROUND OF THE INVENTION

The detection of biologically relevant ligands, subunits and active compounds is of particular interest in the pharmaceutical field, e.g. during development of new drugs. Since many native and synthetic subunits and substrates act as inhibitors of dysfunctional events in the human body, it is of importance to find systems that enable screening or detection of molecules with that mode of action.

In recent years, there has been an exponential increase in the number of compounds that are interesting for screening. Synthetic libraries from drug companies and natural products have been some of the sources of these compounds. The compounds originate from a broad spectrum of different organisms, such as bacteria, insects, plants and marine organisms. This, together with the introduction of combinatorial libraries for the manufacturing of several thousands of compounds have led to a great demand for new screening techniques which are faster and more selective than the ones used today. Known methods used for drug screening are generally based on pure chemical binding between compounds extracted from, for example, natural products and target molecules, such as receptors, enzymes or nucleic acids. The target molecules can also be included in biological systems, such as living cells, where the merits of chemical recognition and biological amplification are combined.

The use of specific target molecules for the evaluation of a compound's biological potential is based on the creation of systems of biological relevance for the analyzed compound. Strategies in this field often include expression of cloned cDNA in different cell systems for the production of a functional target molecule in its natural environment. Typically this involves the need to express and isolate target molecule before they can be used to screen for binding partners or potential drug candidates. Present methods could therefore be improved by automating, and preferably integrating, the step of expressing and isolating the target molecule into the high throughput methods used to screen for binding partners and drug candidates.

### SUMMARY OF THE INVENTION

The present invention provides apparatus, methods and systems for expressing and capturing biomolecules in a single step. Using biochip technology and mass spectrometry, the present invention allows the entire process to be done from small-scale expression mixtures that can be scaled to beyond commonly used 384 well plate formats. Moreover, the incorporation of fluorimetry, mass spectrometry and other advanced methods of detection in the present invention enable the complex interactions between multi-subunit biomolecules to be investigated.

To accomplish these tasks, one embodiment of the present invention is an apparatus for expression and capture of biomolecules. The apparatus comprises at least one reaction vessel defining a reaction space. Within the reaction space is housed an expression system for producing at least some of the biomolecules to be studied. The reaction space also houses a solid support having an adsorbent surface that binds the biomolecules(s) produced by the expression system. At all times during the expression of the biomolecules and their binding to the solid support, the solid support is in fluid communication with the reaction space.

The reaction vessel may be constructed from any material capable of forming and supporting the reaction space. Exemplary materials include plastics, glass, metals, organic fibers, other organic polymers, and the like. Common characteristics of materials suitable for use in constructing the reaction vessel are rigidity and inertness, in the context that the materials comprising the reaction vessel do not contaminate or react with any of the components of the expression system or solid support housed within. Some variations of the apparatus have a single reaction vessel. Other variations have a plurality of reaction vessels, the precise number of reaction vessels being dependent upon the application.

The expression system may comprise cellular components, cell-free systems, or a combination of both to produce the biomolecules to be studied. Exemplary cell based systems include Bacteria (e.g. E. coli), Yeast (e.g. Saccharomyces cerevisiae, and Pichia pastoris), Insect or Insect/viral (e.g., Drosophila melanogaster, Spodoptera frugiperda (Sf9)/Baculovirus), and mammalian (e.g., Chinese Hamster Ovary CHO, and primary hepatocytes). Exemplary cell-free systems include rabbit reticulocyte lysates, canine pancreatic microsomal membranes, E. coli S30 extracts, and wheat germ extracts.

In some aspects of the invention, at least one of the biomolecules comprises a capture moiety. The capture moiety can be an adduct to the biomolecule, an internal sequence or set of sequences that form an epitope specifically recognized by an antibody.

Some aspects of the expression system allow for the expression of more than one biomolecule. In these aspects, the first biomolecule may comprise a capture moiety that binds to the adsorbent surface.

In some aspects, the solid support is not an integral part of the reaction vessel. In these aspects, the solid support may comprise beads, hydrogel, or other suitable inert support. Binding specificity is conferred to the solid support by the inclusion of a specific binding reagent. The specific binding agent may be any suitable agent, including one member of a specific binding pair, such as an antibody, a receptor, an antigen, an enzyme or a receptor ligand. In some alternative aspects, biochips comprise the solid support having a specific binding reagent.

In other embodiments, the solid support is an integral part of the reaction vessel. Alternatives of these embodiments include micro titer plates, ELISA plates, culture dishes and the like, although reaction vessels may be open (no floor, or a porous floor), such as a filter plate, or closed. In those aspects of the invention where the reaction vessel is comprised within a multi-well microtiter plate, the solid support is comprised within a wall and/or floor of each well of the plate.

Regardless of whether the solid support is an integral part of the reaction vessel, certain aspects of these embodiments comprise a plurality of reaction vessels. In those aspects where the solid support comprises or consists of a biochip, the biochip possesses a plurality of addressable locations, each comprising an adsorbent surface corresponding to a separate reaction space. Alternatively, two or more of the addressable locations of the biochip may be in fluid communication with a common reaction space. In some aspects, the biochip is an MS probe.

Another embodiment of the invention is a system for detecting a biomolecule(s). This system comprises at least two components. One component is an apparatus that has at least one reaction vessel defining a reaction space; an expression system housed within the reaction space wherein the expression system expresses at least one biomolecule, and; a solid support having an adsorbent surface that binds the at least one molecule produced by the expression system. The apparatus is constructed to allow the solid support to be in fluid communication with the reaction space. Another component of the system is a detector comprising means for detecting a molecule immobilized on the adsorbent surface of the solid support. The system may also optionally comprise a sonicating device.

In some aspects of the system, the mass-to-charge ratio of the molecules associated with the adsorbent surface is determined using a mass spectrometer. In other aspects molecules associating with the adsorbent surface, or detecting moieties specifically recognizing molecules associating with the adsorbent surface, are fluorescently labeled and are detected using a fluorimeter. Other detectable properties of biomolecules associating with the adsorbent surface include, but are not limited to, absorbance, reflectance, transmittance, birefringence, refractive index, and diffraction. Additional detection techniques include surface plasmon resonance, ellipsometry, resonant mirror techniques, grating coupled waveguide techniques and multipolar resonance spectroscopy.

The invention also includes a method for expressing and capturing a biomolecule(s). This method entails providing the apparatus described above with an expression system for producing biomolecules in the reaction space. The expression system is induced or primed to express the biomolecule(s), which are then captured on the adsorbent surface. The expression system used in the method can be cell-free or cell-based or a combination. Exogenous proteins can optionally be added to supplement those produced by the expression system(s). In those aspects of the method using cell-based expression systems, the apparatus used in the method may include a sonicating device for disrupting the cells. The method can be extended to include the step of detecting or eluting captured biomolecule(s) on the adsorbent surface. In some aspects, the captured biomolecules are eluted prior to detection.

In those aspects of the method where the captured biomolecules(s) is believed to have enzymatic activity, the method further comprises contacting the captured biomolecule with one or more second molecules and detecting evidence of enzymatic activity on the second molecules. The second molecules can be any organic molecules, including biomolecules, and can be introduced into the reaction vessel either through production by one or more expression systems housed in the reaction space, or simply added from exogenous sources, or both. The method can be used to measure any enzymatic activity including, but not limited to, kinase activity, phosphatase activity, glycosylating activity, deglycosylating activity, lipidase activity, delipidase activity, transcriptase activity, DNAase activity, RNAase activity and protease activity.

The method can also be modified to assay modulators of enzyme activity. The modifications necessary comprise contacting the captured biomolecule with an enzymic substrate of the captured biomolecule. The captured biomolecule is then contacted with a plurality of test compounds and evidence of enzymatic activity on the enzymatic substrate is then detected in response to each test compound. Test compounds can be introduced to the biomolecule either before or after introduction of the enzymic substrate.

Another modification that can be made to the method allows for detection of binding partners for the captured molecule. This modification comprises contacting the captured biomolecule with a one or more second biomolecules and then detecting evidence of binding between the captured biomolecule and any of the second biomolecules. The second biomolecules can be introduced ready-made from an exogenous source, or can be produced by expression systems housed within the reaction space. Exemplary biomolecules suitable for capture and analysis using this modification include soluble receptors, membrane bound receptors and antibodies.

The method can be further modified to detect compounds that modulate binding between biomolecules that form a complex. The modification comprises contacting the captured biomolecule with one or more binding partners capable of interacting with the captured biomolecule. The complex is then contacted with one or more test agents before being analyzed for evidence of modulation of complex formation between the captured biomolecule and the binding partner(s). An alternative approach is to contact the captured biomolecule with the test agent(s) prior to introducing the biomolecular binding partners. As with methods previously discussed, the present method can be performed with any biomolecule capable of being captured on the adsorbent surface as a consequence of fused affinity tags or recognized characteristics inherent to the biomolecule itself.

The same approaches to detection can be performed regardless of the variation in methodology used. For example, molecules to be detected can be labeled and detected fluorescently, via radiolabel, affinity tag, enzyme-linked methods that produce a detectable product and the like.

All of the variant methodologies may also be performed using a biochip as an integral, detachable part of the reaction vessel. In some variants of the method, the apparatus used comprises a plurality of addressable locations, each addressable location having an adsorbent surface in fluid communication with a different reaction space. Each of these addressable adsorbent surfaces can be a separate station on a biochip, and in some aspects, the biochip can also serve as an MS probe.

In some aspects of the above-described methods, the captured biomolecules can comprise a capture moiety for binding other molecules. Some methodological aspects comprise a captured biomolecules that is also a detectable moiety.

The present invention also includes kits for the expression and capture of biomolecules. These kits typically comprise an apparatus for the expression and capture of biomolecules as described above, together with instructions, included suggested buffer systems, for its use. Some variations of the kit include buffer systems and optional wash solutions for washing the adsorbent surface. The wash solutions can be ionic interaction modifiers (both ionic strength and pH), a water structure modifier, a hydrophobic interaction modifier, chaotropic reagents or affinity interaction displacers.

Some kit variants also include one or more expression systems for expressing biomolecules of interest, and can include a solid support in the form of a biochip that is an integral, detachable part of the reaction vessel. In a number of variant kits, the biochip provides a plurality of addressable adsorbent surfaces, allowing the apparatus to include a plurality of reaction vessels where the reaction space for each reaction vessel is in fluid communication with a different addressable adsorbent surface of the biochip. Alternatively, one reaction space can be in communication with two or more adsorbent surfaces of the biochip. This latter arrangement allows different biomolecules from the same expression system to be isolated on different adsorbent surfaces in a single step. Throughput can be further enhanced when using mass spectroscopy as a detection device by using a biochip that can double as an MS probe. This arrangement allows adsorbed biomolecules to be readily assayed using SELDI techniques.

In another aspect this invention provides methods for developing a classification algorithm that classifies results of expression/capture experiments performed according to the method of this invention based on the particular expression system used in the experiment. For example, one may perform a first set of experiments in which a first expression system is used that expresses a particular protein, and also perform a second set of experiments in which a second expression system is used that does not express the protein, or that involves the use of a different expression system to express the same protein. This may result in different sets of proteins being expressed and captured on the solid support. A classification system can be developed based on the expression patterns that can distinguish the particular expression system. The methods involve: (a) providing a learning set comprising a plurality of data objects representing expression/capture experiments, wherein the experiments are classified into a plurality of different classes based on type of expression system and wherein each data object comprises data representing specific measurement of a plurality of polypeptides from each experiment captured according to the methods of this invention; and (b) training a learning algorithm with the learning set, thereby generating a classification model, wherein the classification model classifies a data object according to expression system type.

### BRIEF DESCRIPTION OF THE DRAWINGS

FIG. 1 depicts a reaction vessel being open at two ends. The reaction vessel defines a reaction space housing a solid support comprising an adsorbent surface for binding one or more biomolecules. An expression system for producing the one or more biomolecules is introduced at one end. Production of the biomolecule(s) by the expression system occurs within the reaction space. The expressed biomolecule(s) bind to the adsorbent surface of the solid support after which they may be analyzed. In the figure, an exemplary analysis comprising multiple washes is depicted. In this example, each wash is bound to an adsorbent surface of a biochip for further analysis.

FIG. 2 depicts an exemplary reaction vessel assembly. The assembly includes a reaction vessel block “A” that has five bored cylinders. Each cylinder defines the walls of a separate reaction chamber. “C” is a biochip having 5 separate, individually addressable adsorbent surfaces. “B” is a gasket that interfaces with both block “A” and biochip “C”, preventing leakage and cross-contamination between reaction vessels. Block “D” is an assembly stand that allows all pieces of the assembly to be fastened together as a unit.

FIG. 3 depicts an exemplary reaction vessel assembly similar to that described in FIG. 2, but in this embodiment all five adsorbent surfaces are in fluid communication with a common reaction space. This arrangement allows multiple different biomolecules produced by an expression system to bind to separate adsorbent surfaces based on the nature of the adsorbent surface bound, and the nature of the capture moiety of the respective biomolecules.

FIG. 4 depicts a biochip comprising a plurality of adsorbent surfaces. The biochip has six different adsorbent locations, each individually addressable, and classified according to a basis of attraction (hydrophobic, ionic, coordinate covalent and mixed function). The biochip has several positions for each type of adsorbent, allowing interrogation of the separate positions at different times with different eluants, or for archiving and subsequent analysis.

FIG. 5 depicts a reaction vessel assembly similar to that depicted in FIG. 1. The cartoon depicts an expression system for two biomolecules that interact with each other to form a biomolecular complex. One of the two biomolecules has a capture moiety that is recognized by the adsorbent surface of the biochip. The cartoon shows the two biomolecules interacting to form a complex and the complex being captured by the adsorbent surface via the capture moiety of one of the biomolecules (stage “D)” in the figure). The captured complex can then be washed with a solution comprising one or more components affecting complex interaction (stage “E)” in the figure). The nature of the wash couple with the effect the wash has on complex association will provide an indication of the types of molecular forces involved in holding the complex together. Please note that FIG. 5 is by way of example and should not be construed as limiting on the invention. Other modes of use for the invention are contemplated and many of these are described herein.

FIG. 6 depicts four panels, including exemplary results achieved using the present invention. Panel FIG. 1 depicts a multiwell filter plate/biochip reaction vessel assembly. Panel FIG. 2 depicts exemplary expression results using the present invention having a bacterial expression system induced by IPTG. Panel FIG. 3 depicts expression results using the system described for panel FIG. 2, but with protein release occurring after lysozyme treatment. Panel FIG. 4 is additional expression results achieved using the bacterial expression system described for panel FIG. 2, the results illustrating the differences achieved using different protein isolation techniques.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “addressable location” refers to a position that can be fixed in space within defined limits and accessible through an index unique for the position.

“Adsorbent” or “capture reagent” refers to any material capable of binding an analyte (e.g., a target polypeptide). “Chromatographic adsorbent” refers to a material typically used in chromatography. Chromatographic adsorbents include for example, ion exchange materials, metal chelators, hydrophobic interaction adsorbents, hydrophilic interaction adsorbents, dyes, and mixed mode adsorbents (e.g., hydrophobic attraction/electrostatic repulsion adsorbents). “Biospecific adsorbent” refers to an adsorbent comprising a biomolecule, e.g., a nucleotide, a nucleic acid molecule, an amino acid, a polypeptide, a simple sugar, a polysaccharide, a fatty acid, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than a chromatographic adsorbent. Further examples of adsorbents for use in SELDI can be found in U.S. Pat. No. 6,225,047 (Hutchens and Yip, “Use of retentate chromatography to generate difference maps,” May 1, 2001).

“Adsorb” refers to the detectable binding between an absorbent and an analyte either before or after washing with an eluant (selectivity threshold modifier).

“Biochip” refers to a solid substrate having a generally planar surface to which a capture reagent is attached (the capture reagent can be an inorganic, organic, or biologic moiety). Biochips, thus, comprise an “adsorbent surface.” Frequently, the surface of the biochip comprises a plurality of addressable locations, each of which location has the capture reagent bound there.

Upon capture, analytes can be detected by a variety of detection methods including for example, gas phase ion spectrometry methods, optical methods, electrochemical methods, atomic force microscopy and radio frequency methods. Gas phase ion spectrometry methods are described herein. Of particular interest is the use of SELDI, a mass spectrometric method in which analytes are captured on the surface of a biochip and detected by, e.g., laser desorption/ionization mass spectrometry. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); International publication WO 00/04389 (Wagner et al., “Arrays of protein-capture agents and methods of use thereof,” Jul. 27, 2000) and International publication WO 00/56934 (Englert et al., “Continuous porous matrix arrays,” Sep. 28, 2000).

“Surface-Enhanced Neat Desorption” or “SEND” is a version of SELDI that involves the use of probes (“SEND probe”) comprising a layer of energy absorbing molecules attached to the probe surface. Attachment can be, for example, by covalent or non-covalent chemical bonds. Unlike traditional MALDI, the analyte in SEND is not required to be trapped within a crystalline matrix of energy absorbing molecules for desorption/ionization. “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer comprising α-cyano-4-methacryloyloxycinnamic acid and octadecylmethacrylate (“C18 SEND”). SEND is further described in U.S. Pat. No. 5,719,060 and WO 03/64594 (Kitagawa, “Monomers And Polymers Having Energy Absorbing Moieties Of Use In Desorption/Ionization Of Analytes”, Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and desorption without the need to apply external matrix. The C18 SEND biochip is a version of SEAC/SEND, comprising a C18 moiety which functions as a capture reagent, and a CHCA moiety which functions as an energy absorbing moiety.

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, IMAC-3, LSAX-30, LWCX-30, IMAC-40, PS-10 and PS-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, IMAC-3, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol) methacrylate for hydrophobic binding. The SAX-2 biochip has quarternary ammonium functionalities for anion exchange. The WCX-2 biochip has carboxylate functionalities for cation exchange. The IMAC-3 biochip has copper ions immobilized through nitrilotriacetic acid or IDA for coordinate covalent bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The LSAX-30 (anion exchange), LWCX-30 (cation exchange) and IMAC-40 (metal chelate) biochips have functionalized latex beads on their surfaces. Such biochips are further described in: WO 00/66265 (Rich et al. (“Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application Ser. No. 09/908,518 (Pohl et al., “Latex Based Adsorbent Chip,” Jul. 16, 2002) and U.S. patent application 60/350,110 (Um et al., “Hydrophobic Surface Chip,” Nov. 8, 2001).

“Biomolecule”, in the context of the present invention, includes any molecular species synthesized during the course of chemical reactions related to the expression systems of the invention, or any molecular species that could be formed as an intermediate or product of a metabolic process. By “metabolic process” is meant any chemical reaction or interaction initiated by a living cell, a cellular organism, the protoplasm of a cell or cellular organism, or purified (including partially purified) components derived from a cell or cellular organism. Biomolecules and metabolic processes need not exist within the living cell or cellular organism, and include extracellular reactions brought about by cellular activity.

“Buffer system” refers to a solution capable of accepting an influx of acidic or basic components without appreciable change in the pH of the system. By “without appreciable change” is meant that the pH value does not change more than one pH unit, preferably not more than 0.6 of a unit, most preferably not more than 0.3 of a unit.

“Capture moiety” refers to a composition that can specifically bind to certain types of adsorbent surfaces comprising a complementary binding partner for the particular capture moiety used. The chemistry involved in the binding reaction between a capture moiety and an adsorbent surface is dependent upon the nature of the capture moiety/adsorbent pair used. For example, hexahistidine sequences added to a polypeptide or protein chelate to adsorbents comprising nickel atoms, FLAG sequences are recognized and bound noncovalently by FLAG-specific antibodies, and adsorbent surfaces comprising receptors or enzymes can specifically bind capture moieties comprising their respective ligands and substrates, or homologues thereof. Typically, capture moieties are covalently attached to proteins, and serve as a means for anchoring the proteins to an adsorbent surface.

“Cell-free” refers to systems capable of producing biomolecules in the absence of intact cells or organisms. Exemplary cell-free systems include rabbit reticulocyte lysates, canine pancreatic microsomal membranes, E. coli S30 extracts, and wheat germ extracts.

“Complex” or “multi-subunit complex” refers to the association of two or more biomolecules, forming a discrete aggregate.

“Detectable moiety” or a “label” refers to a composition detectable by spectroscopic, photochemical, biochemical, immunochemical, or chemical means. For example, useful labels include .sup.32 P, .sup.35 S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantitate the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavadin. The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavadin, or a nucleotide sequence, which is the binding partner for a complementary sequence, to which it can specifically hybridize. The binding partner may be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., P D. Fahrlander and A. Klausner, Bio/Technology (1988) 6:1165.) Quantitation of the signal is achieved by, e.g., scintillation counting, densitometry, or flow cytometry.

“Expression systems” can be cell-based or cell-free, as defined herein, and serve to produce proteins and protein-based products from the nucleic acids encoding them.

Two or more chambers that are said to be in “fluid communication” share a common connection through which a liquid or gas may flow. Although flow control devices may exist along the path between chambers that are in fluid communication, and may regulate flow rates or passage, the chambers are said not to be in fluid communication during times or points where flow or passage is completely blocked.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Hydrogel” refers to a colloid in which the particles are in the external or dispersion phase and water in the internal or dispersed phase.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrapole filter, ion trap, ion cyclotron resonance, electrostatic sector analyzer and hybrids of these. “Mass spectrometry” refers to the use of mass spectrometry to detect gas phase ions.

“Ion source” refers to a sub-assembly of a gas phase ion spectrometer that provides gas phase ions. In one embodiment, the ion source provides ions through a desorption/ionization process. Such embodiments generally comprise a probe interface that positionally engages probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) fast atoms (used in fast atom bombardment); (2) high energy particles generated via beta decay of radionucleotides (used in plasma desorption); and (3) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. Typically, a sample is placed on the surface of a probe, the probe is engaged with the probe interface and the probe surface is struck with the ionizing energy. The energy desorbs analyte molecules from the surface into the gas phase and ionizes them. Other forms of ionizing energy for analytes include, for example: (1) electrons which ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device that can be used to introduce ions derived from an analyte into a gas phase ion spectrometer, such as a mass spectrometer. A “probe” will generally comprise a solid substrate (either flexible or rigid) comprising a sample-presenting surface on which an analyte is presented to the source of ionizing energy. “SELDI probe” refers to a probe comprising an adsorbent (also called a “capture reagent”) attached to the surface. “Adsorbent surface” refers to a surface to which an adsorbent is bound. “Chemically selective surface” refers to a surface to which is bound either an adsorbent or a reactive moiety that is capable of binding a capture reagent, e.g., through a reaction forming a covalent or coordinate covalent bond.

“SELDI MS probe” refers to a probe comprising an adsorbent (also called a “capture reagent”) attached to the surface.

“Solid support” refers to any insoluble surface including beads or plastic strips. The term also refers to a solid phase to which an adsorbent is attached or deposited.

“Specific binding reagent” refers to any first composition that recognizes and binds to a second composition in a manner that is determinative of the presence of the second composition in a heterogeneous population of molecules. Thus, under designated conditions, the first composition binds to the second composition at least two times the background and does not substantially bind in a significant amount to other molecules present in the sample.

In the case of specific binding reagents that are antibodies, specific binding may require selection of an antibody for its specificity. For example, polyclonal antibodies raised to Ras protein from specific species such as rat, mouse, or human can be selected to obtain only those polyclonal antibodies that are specifically immunoreactive with Ras protein and not with other proteins, except for polymorphic variants and alleles of Ras protein. This selection may be achieved by subtracting out antibodies that cross-react with Ras proteins from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Wash solution” or “eluant” refers to a liquid that can be used to wash and remove unbound material from an adsorbent surface.

### DETAILED DESCRIPTION

I. Introduction

The present invention provides apparatus and methods for the expression and analysis of biomolecules in a single step. This is accomplished by expressing one or more biomolecules of interest in a reaction vessel that is in fluid communication with an adsorbent surface that recognizes the biomolecule(s). Recognition by the adsorbent surface is assured by including in the expressed biomolecule a capture moiety compatible with the adsorbent surface.

In addition to the expression system and the solid support, the reaction vessel may contain other components such as nutrients for the expression system (when cell-based), expression-inducing agents, modulators of protein binding or activity, and other components necessary for the application being pursued.

Using the apparatus and methods described herein, molecular complexes can be studied; including the nature of molecular components making up the complex and characteristics of their interactions. Some embodiments allow for the screening of combinatorial libraries. Other embodiments allow for isolation and identification of ligands and other binding partners.

Finally, the invention provides kits comprising various components of the invention described herein. The kits aid in performing the techniques of the invention by conveniently providing instruction and control reagents for the correct operation of the apparatus and methods contained therein.

II. Expression and Capture Apparatus

The apparatus of the present invention comprise a reaction vessel defining a reaction space that houses an expression system and is simultaneously in fluid communication with a solid support having a binding moiety specific for at least one of the products of the expression system. This arrangement allows proteins produced by the expression system to be captured on the solid support in one-step using a single device.

A. Reaction Vessel

The reaction vessel defines the reaction space; the location of the expression systems producing biomolecules of interest, and the solid support comprising the adsorbent surface(s) for capturing those biomolecules. The reaction vessel can be any shape, preferably cylindrical, defining a reaction space having a volume of between 0.01 and 10 ml, preferably between 0.02 and 1 ml, most preferably between 0.05 and 0.5 ml. The reaction space can be open or closed. For example, each well of a microtitre plate can serve as a closed reaction vessel with the reaction space being defined by the floor and cylindrical wall of the well. A similar closed reaction vessel can be formed using a cell culture plate.

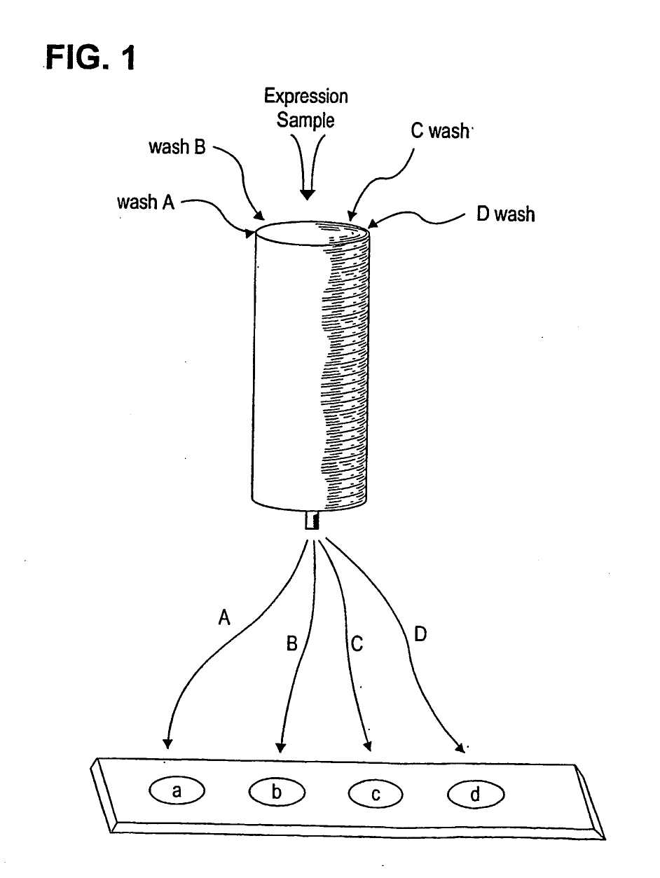

An example of an open reaction vessel is a column body, such as that depicted in FIG. 1. The reaction space within an open reaction vessel preferably has a volume of less than 20 ml, more preferably less than 10 ml, preferentially less than 1 ml and most preferably 0.5 ml or less. Open reaction vessels can be operated in batch mode, or as flow devices supporting laminar or turbulent flow characteristics.

Referring to FIG. 1, the expression system is introduced into the reaction space from a first end of the reaction vessel. Also housed in the reaction space is a solid support comprising a specific binding moiety for at least one of the products of the expression system. Within the reaction vessel, the expression system is maintained in a state supporting expression of the desired products. The desired products are allowed to bind to the solid support, which is then washed with elution solutions comprised of different eluting agents. The eluate produced using each elution solution is then analyzed for its content using, for example a biochip as depicted in FIG. 1. In addition to knowledge of the elution conditions, the additional analytical data produced from these analyses can be used to elucidate properties of proteins recovered including molecular weight, hydrophobicity, isoelectric point, and, in the case of multisubunit complexes, the chemical nature of intramolecular interactions. Other examples of open reaction vessels include multi-well filter plates and funnels having sintered glass or porous plastic bases.

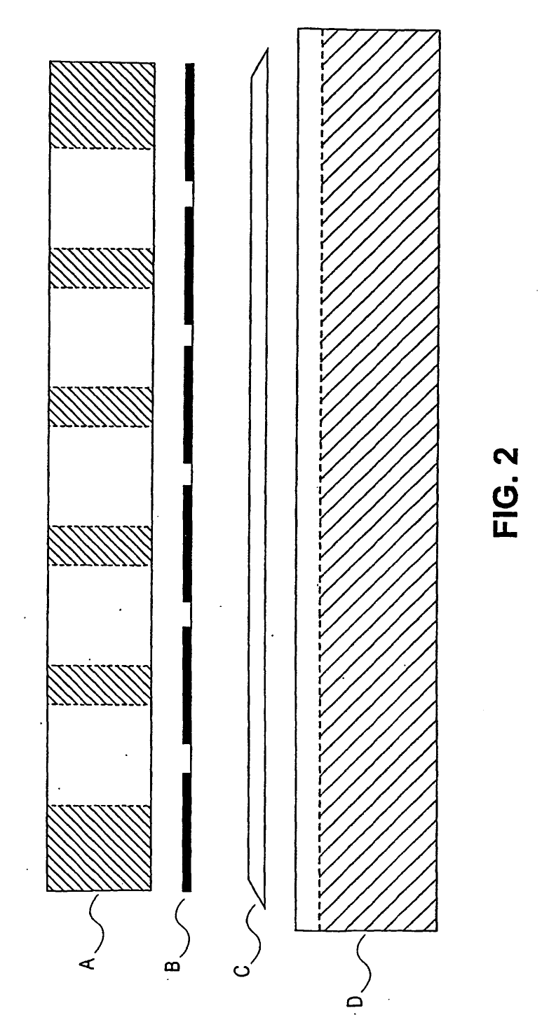

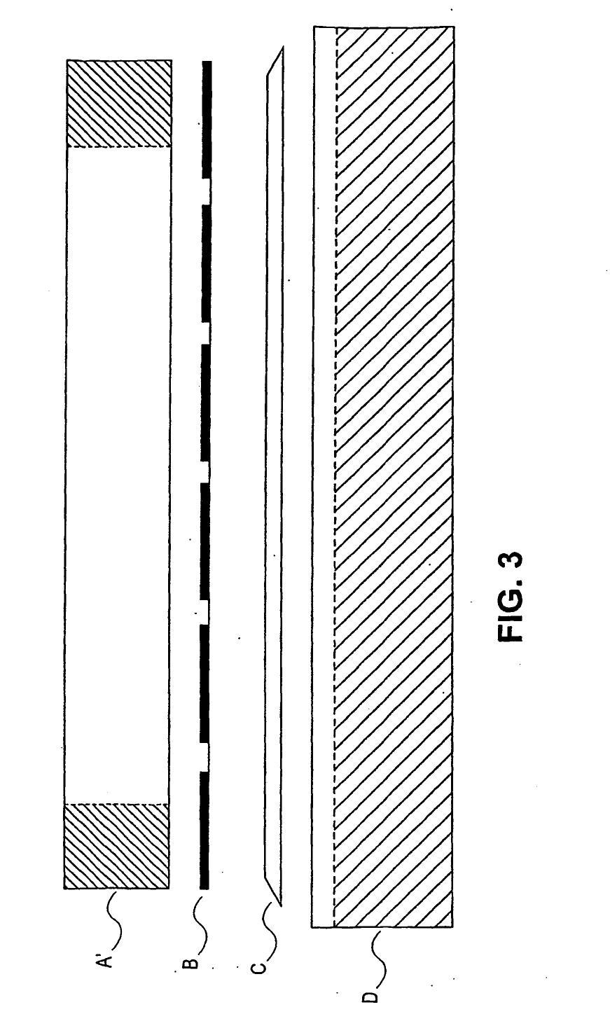

Reaction vessels need not consist of a single unit, but may also be constructed from multiple components. For example, FIG. 2 depicts a closed reaction vessel of the present invention that comprises a biochip with a plurality of adsorbent surfaces, each adsorbent surface in fluid communication with a reaction chamber. Referring to FIG. 2, “A” is a housing comprising a series of reaction vessels; “C” is the biochip with adsorbent surfaces a, b, c, d, and e; “B” is a gasket providing a seal between parts “A” and “C”, thereby preventing leakage of the contents of the reaction vessels when the device is assembled; and “D” is a housing that aids in maintaining the assembled device when in use. Fluid communication between the adsorbent surfaces of the biochip and the reaction space is maintained by the presence of channels in the gasket. The channels may be of any dimension up to or within the dimensions defined by the cross-section of the reaction space. Typically, a single channel exists for each adsorbent surface/reaction vessel pair.

In the device depicted in FIG. 2, each reaction vessel may house a different expression system, but this should not be construed as a limitation of the invention. As exemplified in FIG. 3, the reaction vessel may be constructed in a manner that allows two or more solid supports to be in simultaneous fluid communication with a common reaction space. Such an arrangement allows the product(s) of a single expression system to be captured on multiple solid supports in a single step with, for example, each solid support having a different specific binding moiety. The product(s) captured on each solid support can then be analyzed further as described herein.

B. Expression Systems

A unique aspect of the present invention is the ability to express and capture biomolecules in a single step, thereby facilitating isolation and analysis of biomolecules of interest. Suitable expression systems for this purpose may be cell-free, cell based or a combination of both. Cell-based expression systems may include cells from any source, and either prokaryotic or eukaryotic or both. The cells used may also be recombinant in nature, express native cellular proteins, or comprise a combination of both.

The present invention also contemplates embodiments comprising multiple expression systems and single expression systems expressing multiple biomolecules. Such embodiments are useful in producing components of multi-subunit complexes, receptor-ligand pairs or enzyme-substrate combinations. Similarly, such embodiments allow screening of nucleic acid libraries encoding putative components of biological systems for relevant activity of function by creating recombinant expression systems for the components to be studied and incorporating those expression systems into the devices and methods of the present invention.

Regardless of the exact nature of the expression system, biomolecule expression occurs within the reaction space of the present invention. Expression within the reaction space however does not preclude the addition of solutions, suspensions and mixtures of molecules produced outside the expression system. For example, in the study of molecular interactions, the biomolecular composition produced by the expression system can be supplemented with putative binding partners for the expressed biomolecules. Another example is the addition of putative small molecule effectors of the expressed biomolecules to ascertain which small molecule effectors modulate the activity or structure of the expressed biomolecules. Added molecular components may also be affinity-tagged or labeled with a detectable marker as described herein to aid in carrying out the particular application being pursued. Techniques such as these find wide use in screening compound libraries and in the study of multi-molecular interactions.

Growth factors, nutrients, selection agents and other components normally associated with cell culture techniques may also be added to the reaction vessel as needed to support the expression systems therein. The housing of the reaction vessel may optionally comprise a heating or cooling element to maintain the contents of the reaction space at a constant temperature. The reaction vessel housing may also optionally comprise lighting, atmospheric sensors, gas vents and/or agitating devices, such as orbital mixtures, to promote thorough distribution of nutrients and waste to maintain a desirable atmosphere for the expression of biomolecules.

A wide variety of biomolecules may be produced by the expression systems of the present invention. These include, but are not limited to, enzymes, receptors, receptor ligands, enzyme substrates, structural molecules, and hormones including paracrine factors, ion channels, antibiotics, and cell markers. Expression of the subject biomolecules may be inducible or constitutive, depending upon the particular application.

Typically, at least one of the expressed biomolecules is affinity tagged or otherwise possesses a capture moiety, as defined herein. The capture moiety allows the biomolecule to be recognized by, and bound, to an adsorbent surface of the invention. By way of example, the capture moiety can take the form of a fusion adduct, such as a his-tag, Flag or other epitope sequence. Alternatively the affinity tag can be a sequence tag present in the primary structure of the biomolecule itself, such as a specific nucleic acid or amino acid sequence, or a characteristic glycosylation pattern defining an epitope or other characteristic specific for the biomolecule and capable of recognition by a capture moiety. Additional examples and mechanisms for tagging and capturing biomolecules are described herein below.

In some embodiments, expressed biomolecules may also be labeled with a detectable marker. Labeling is typically accomplished by expression of a fusion construct comprising the biomolecule of interest and the detectable marker. Exemplary detectable markers include fluorescent proteins, epitope tags and enzymes capable of converting a substrate into a detectable product (e.g., β-galactosidase).

1. Cell-Free Expression Systems

Use of cell-free expression systems minimizes potential contamination of the biomolecular product of interest by drastically reducing the number of components present in the expression system, when compared to cell-based systems. Embodiments of the present invention may comprise cell-free expression systems in liquid phase, or with components attached to a solid support, or both. Attaching the cell-free system to a solid support has the benefit of retaining the components of the expression system in the reaction space. By preventing components of the expression system from leaving the reaction space, the components are prevented from migrating with and contaminating the biomolecule products of the expression system. Techniques for immobilizing expression systems are known in the art (See e.g., Klibanov, A. M. (1983). Immobilized Enzymes and Cells as Practical Catalysts. Science 219, 722-727).

Numerous cell-free expression systems compatible with the present invention are known in the art. see Anderson, C. W., Straus, J. W. and Dudock, B. S. (1983). Preparation of a Cell-free Protein-synthesizing System from Wheat Germ is described in Methods Enzymol. 101, 635-644; Chambliss, G. H., Henkin, T. M. and Leventhal, J. M. (1983). Bacterial in vitro Protein-synthesis Systems are described in Methods Enzymol. 101, 598-605; Merrick, W. C. (1983). Translation of Exogenous mRNAs in Reticulocyte Lysates can be found in Methods Enzymol. 101, 606-615. For nucleic acids provided in the form of DNA, the expression can be carried out in a medium containing a coupled transcription/translation system. See e.g., Chen, H.-Z. and Zubay, G. (1983). Prokaryotic Coupled Transcription-translation. Methods Enzymol. 101, 674-690; Bujard, H., Gentz, R., Lanzer, M., Stueber, D., Mueller, M., Ibrahimi, I., Haeuptle, M.-T. and Dobberstein, B. (1987). A T5 Promoter-based Transcription-translation System for the Analysis of Proteins in vitro and in vivo. Methods Enzymol. 155, 416-433; Tymms, M. J. and McInnes, B. (1988). Efficient in vitro Expression of Interferon Analogs Using SP6 Polymerase and Rabbit Reticulocyte Lysate. Gene Anal. Tech. 5, 9-15; Baranov, V. I., Morozov, I. Yu., Ortlepp, S. A. and Spirin, A. S. (1989). Gene Expression in a Cell-free System on the Preparative Scale, Gene 84, 463-466; Lesley, S. A, Brow, M. A. and Burgess, R. R. (1991). Use of in vitro Protein Synthesis from Polymerase Chain Reaction-generated Templates to Study Interaction of Escherichia coli Transcription Factors with Core RNA Polymerase and for Epitope Mapping of Monoclonal Antibodies. J. Biol. Chem. 266, 2632-2638. Commercial products for cell-free translation of nucleic acids are also available. For example, a coupled transcription-translation reaction kit based on a reticulocyte lysate system can be purchased from Promega (Promega TNT™).

2. Cell-Based Expression Systems

Cell-based expression systems, particularly eukaryotic expression systems, are often the preferred method for producing biomolecules of interest because such systems allow for proper post-translational modifications of expressed eukaryotic (particularly mammalian) polypeptides to occur. In particular, eukaryotic cells that possess the cellular machinery for proper processing of the primary transcript, glycosylation, phosphorylation, and advantageously, plasma membrane insertion of a polypeptide may be used as host cells. Post-translational modifications are frequently important to biomolecular function, but often impossible to perform in a workable cell-free system. Cell-based expression systems may be practiced in suspension or attached to a solid support (See e.g., Klibanov, A. M. (1983). Immobilized Enzymes and Cells as Practical Catalysts. Science 219, 722-727), with the same benefits as discussed for immobilized cell-free systems above. In instances where the biomolecule of interest is not excreted by the production cell, the cell can be disrupted to allow release of the biomolecule. Methods disrupting cells are well known in the art and include such procedures as osmotic shock, detergent treatment, Dounce homogenization and sonication and enzyme treatment. In some embodiments of the present invention, the device for single step expression and capture of biomolecules comprises a sonicator to aid cell disruption allowing release of the biomolecule of interest.

a. Production Cells

Production cells of the expression system may express recombinant or native biomolecules and can be derived from prokaryotes or eukaryotes, depending upon the desired biomolecular expression product. Numerous cell lines and cultures are available for use as production cells, and many may be obtained through the American Type Culture Collection (ATCC), which is an organization that serves as an archive for living cultures and genetic materials (www.atcc.org). Again, depending upon the application and the product desired, production cells may be obtained from any source including skin, bone, neuron, axon, cartilage, blood vessel, cornea, muscle, fascia, brain, prostate, breast, endometrium, lung, pancreas, small intestine, blood, liver, testes, ovaries, cervix, colon, skin, stomach, esophagus, spleen, lymph node, bone marrow, kidney, peripheral blood, embryonic or ascite cells, and all cancers thereof. An appropriate production cell can be determined by one of skill in the art based on the vector constructs at hand and the desired result. Bacterial cells used as production cells include DH5α, JM109BL21, and KC8, as well as a number of commercially available bacterial lines such as SURE™, Competent Cells and Solopack™. Gold Cells (Stratagene™ La Jolla). Alternatively, bacterial cells such as strain of E. coli (e.g., LE392) could be used. Other exemplary bacterial production cells include gram-positive bacteria (Palva et al., Gene, 22:229-235 (1983); Mosbach et al., Nature, 302:543-545 (1983), and gram-negative bacteria such as Escherichia coli (cf. Sambrook et al., supra). Examples of suitable yeast cells include Saccharomyces sp. or Schizosaccharomyces sp. Other suitable fungal sources include Aspergillus sp., Neurospora sp., Fusarium sp. or Trichoderma sp., in particular strains of A. oryzae, A. nidulans, A. niger, or Pichia pastoris.

Examples of eukaryotic production cells for replication and/or expression of a vector include HeLa, NIH3T3, Jurkat, 293, Cos, CHO, Saos, WelH, baby rat kidney (BRK) cell lines, insect cell lines, bird cell lines, and PC12. Primary cell lines are also contemplated for use with this invention.

Freshney (Culture of Animal Cells, a Manual of Basic Technique, third edition Wiley-Liss, New York (1994)) and the references cited therein provide a general guide to the culture of cells. Transduced cells are cultured by means well known in the art. See, also Kuchler et al. (1977) Biochemical Methods in Cell Culture and Virology, Kuchler, R. J., Dowden, Hutchinson and Ross, Inc. Mammalian cell systems often will be in the form of monolayers of cells, although mammalian cell suspensions are also used.

b. Recombinant Systems

For those embodiments of the present invention comprising expression systems having recombinant genetic constructs, including systems expressing viral proteins or comprising viral-derived genetic constructs, this invention relies on routine techniques in the field of recombinant genetics. Basic texts disclosing the general methods of use in this invention include Sambrook et al., Molecular Cloning: A Laboratory Manual (2^{nd} ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (Kb) or base pairs (bp). These are estimates derived from agarose or acrylamide gel electrophoresis, from sequenced nucleic acids, or from published DNA sequences. For proteins, sizes are given in kilodaltons (kDa) or the number of amino acid residues. Proteins sizes are estimated from gel electrophoresis, from automated protein sequencing, from derived amino acid sequences, or from published protein sequences.

Oligonucleotides that are not commercially available can be chemically synthesized according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Purification of oligonucleotides may be performed by either native acrylamide gel electrophoresis or by anion-exchange HPLC as described in Pearson & Reanier, J. Chrom., 255:137-149 (1983).

The sequence of cloned genes and synthetic oligonucleotides can be verified after cloning using, e.g., the chain termination method for sequencing double-stranded templates of Wallace et al., Gene, 16:21-26 (1981).

c. Promoters

Both constitutive and inducible expression systems are contemplated by the present invention. Whether a given expression system constitutively or inducibly produces a desired biomolecule is largely dependent on the promoter element(s) controlling gene transcription. Suitable promoters for the present invention include any constitutive or inducible promoter that can be expressed in the particular cell type used in the present invention. Those skilled in the art know that the choice of the promoter depends upon the type of production cell to be employed for expressing a gene(s) under the transcriptional control of the chosen promoter. A wide variety of promoters functional in viruses, prokaryotic cells and eukaryotic cells are known in the art and may be employed in the present invention.

Exemplary constitutive promoters in mammals include the EF-1α promoter, viral promoters such as HSV, TK, RSV, SV40 and CMV promoters, various housekeeping gene promoters, as exemplified by the β-actin promoter. Examples of suitable mammalian inducible promoters include promoters from genes such as cytochrome P450 genes, metallothionein genes, hormone-inducible genes, such as the estrogen gene promoter, and such like. Promoters that are activated in response to exposure to ionizing radiation, such as fos, jun and erg-1, are also contemplated.

Exemplary plant promoters include: the CaMV 35S promoter (Odell, J. T., Nagy, F., Chua, N. H., Nature, 313:810-812 (1985)), the CaMV 19S (Lawton, M. A., Tierney, M. A., Nakamura, I., Anderson, E., Komeda, Y., Dube, P., Hoffman, N., Fraley, R. T., Beachy, R. N., Plant Mol. Biol., 9:315-324 (1987)), nos (Ebert, P. R., Ha, S. B., An. G., PNAS, 84:5745-5749 (1987)), Adh (Walker, J. C., Howard, E. A., Dennis, E. S., Peacock, W. J, PNAS, 84:6624-6628 (1987)), sucrose synthase (Yang, N. S., Russell, D., PNAS, 87:4144-4148 (1990)), α-tubulin, actin (Wang, Y., Zhang, W., Cao, J., McEhoy, D. and Ray Wu., Molecular and Cellular Biology, 12:3399-3406 (1992)), cab (Sullivan, T. et al., Mol. Gen. Genet, 215:431-440 (1989)), PEPCase (Hudspeth, R. L. and J. W. Grula., Plant Mol. Biol., 12:579-589 (1989)) or octopine synthase (OCS) promoters, the light-inducible promoter from the small subunit of ribulose bis-phosphate carboxylase (Khoudi, et al., Gene, 197:343 (1997)) and the mannopine synthase (MAS) promoter (Velten et al., EMBO J., 3:2723-2730 (1984); Velten & Schell, Nucleic Acids Research, 13:6981-6998 (1985)).

3. Expressed Biomolecules

As noted above, expressed biomolecules of the present invention can be from any source, and perform any function. Enzymes, receptors, receptor ligands, membrane channels, structural molecules, lipids, hormones, sugars (both complex and simple) are some of the molecular classes constituting biomolecules, with enzymes and receptors comprising preferred embodiments. Exemplary enzymes that can be studied using the present invention include kinases, phospatases, glycosylases, glycosidases, proteases, lipases, lipid synthase, lipases, polymerases, DNAases and RNAases.

A given expression system may produce one or several biomolecules of interest, and the reaction space may hold any number of expression systems necessary to perform the desired application. These approaches lend themselves to studies of intermolecular reactions, including complex formation, enzyme catalysis and ligand binding. For example, the interaction of subunits in a complex consisting of two proteins can be studied by expressing both proteins in the same reaction space, as described in detail below.

Typically at least one of the expressed biomolecules comprises a capture moiety that can be used to bind the biomolecule specifically to an adsorbent surface of the invention as discussed in detail elsewhere in this application. Biomolecules may also optionally comprise a detectable moiety. Both capture and detectable moieties are discussed in detail below.

a. Harvesting Intracellular Biomolecules

In cell-based systems, biomolecular synthesis frequently results in a product that is not excreted from the production cell. For those biomolecules not routinely excreted by the production cell, some method must be provided to harvest the biomolecule from the cell, so that it may be contacted to the adsorbent surface of the solid support. To accomplish this task, the present invention optionally comprises methods and devices for permeating or otherwise disrupting the cell membrane of the production cell.

For example, the reaction vessel may optionally comprise a sonicating device. Sonicating devices produce high frequency waves that cause cell membranes to rupture, causing the contents of the cell to be released. Methods for disrupting cells to release intracellular proteins include osmotic shock, detergent treatment and others described in e.g., Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series.

Alternatively, the present invention also provides methods for attaching “secretion tags” to biomolecules normally retained in the cell secretion tags trick the cell into secreting the tagged protein. This technique is discussed in J. Biol. Chem., 267, 4882-4888, 1992, and improved upon by Udaka et al., Nippon Nogeikagaku Kaishi, 67(3), 372, 1993).

b. Capture Moiety Tags

At least one biomolecule of the invention includes a capture moiety tag. Capture moiety tags perform a number of functions in the present invention. For example, capture moiety tags bind the tagged molecule to an adsorbent surface, aiding isolation of the molecule. In addition, they provide a means of identifying the tagged molecule using labeled binding agents that specifically recognize the capture moiety. Exemplary capture moieties include epitope and his-tags, which are attached to the biomolecule to be captured to form a fusion protein. In these instances, a cleavable linker sequence, such as those specific for Factor XA or enterokinase (Invitrogen, San Diego, Calif.) may be optionally included between the biomolecule and the capture moiety to facilitate isolation and/or separation of the components of the fusion molecule. Protein domains specifically recognized by designer ligands may also be used as capture moieties (See, e.g., Deisenhofer, J., Biochemistry 20 (1981) 2361-2370). Many other equivalent capture moieties are known in the art. See, e.g., Hochuli, Chemische Industrie, 12:69-70 (1989); Hochuli, Genetic Engineering, Principle and Methods, 12:87-98 (1990), Plenum Press, N.Y.; and Crowe, et al. (1992) OIAexpress: The High Level Expression & Protein Purification System, QIAGEN, Inc. Chatsworth, Calif.; which are incorporated herein by reference. Antigenic determinants and other characteristic properties of the biomolecule to be adsorbed may also serve as capture moiety tags, as described below.

Epitope Tagging

Epitope tags consist of an amino acid sequences that allow affinity recognition and specific binding of the tagged molecule by an antibody raised against the tag peptide. Thus, by including an epitope tag on the biomolecule, the biomolecule can be specifically isolated from a complex mix. By isolating the biomolecule from other cellular and media components, detection fidelity and sensitivity can be enhanced. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); a α-tubulin epitope peptide (Skinner et al., J. Biol. Chem., 266: 15163-15166 (1991)); and the T7 gene 10 protein peptide tag (Lutz-Freyermuth et al., Proc. Natl. Acad. Sci. USA, 87: 6393-6397 (1990)).

His-Tag

Biomolecules with a 6×His tag bind to Ni-NTA solid supports with high affinity allowing the biomolecule to be isolated in a simple one-step procedure (for details see: The QIAexpressionist (1995) QIAGEN, Inc., Chatsworth, Calif.). When a protein that has been “his-tagged” is placed on the nickel column, the histidine residues form a chelate complex with the nickel bound to the column, immobilizing the tagged biomolecule. The his-tagged biomolecule can be released from the Ni-NTA support with nickel chelating agents. Imidazole is typically used for this purpose. Other chelating structures, such as IDA, CMA and TED can be used in analogous methods, known by those of ordinary skill in the art.

Native Sequence Tags

Biomolecules may possess native structures that can be recognized by adsorbent surfaces, either specifically or non-specifically, alleviating the need for recombinant tag motifs such as epitope and his tags. Suitable native structures include antigenic determinants, polysaccharide structures of glycoproteins, binding pockets for specific ligands, overall physical characteristics such as overall hydrophobicity or charge, or any other characteristic of the biomolecule that allows selection of an adsorbent surface that will recognize and adsorb it. Sequence tags can associate with an adsorbent surface via any molecular attractive force, or combination thereof that can be formed between a biomolecule and an adsorbent surface. These forces include van der Waals, ionic, covalent hydrophobic, hydrogen bonding and others mentioned below in relation to adsorbent surfaces.

c. Detectable Moiety Tags

The particular detectable moiety used in the assay is not a critical aspect of the invention, as long as it does not significantly interfere with the specific binding or functional aspects of the biomolecules used in the assay. The detectable moiety can be any material having a detectable physical or chemical property. Such detectable labels have been well developed and, in general, most any label can be applied to the present invention. Thus, a detectable moiety is any composition detectable by spectroscopic, photochemical, biochemical, immunochemical, and electrical, optical or chemical means. Useful detectable moieties in the present invention include magnetic beads (e.g., DYNABEADS™); fluorescent dyes and proteins, and techniques capable of monitoring the change in fluorescent intensity, wavelength shift, or fluorescent polarization (e.g. fluorescein isothiocyanate, Texas red, rhodamine, and the like); radiolabels (e.g., ³H, ¹²⁵I, ³⁵S, ¹⁴C, or ³²P); enzymes (e.g., horse radish peroxidase, alkaline phosphatase and others commonly used in an ELISA); and colorimetric labels such as colloidal gold or colored glass or plastic beads (e.g. polystyrene, polypropylene, latex, etc.). For exemplary methods for incorporating such detectable moieties, see U.S. Pat. Nos. 3,940,475; 3,817,837; 3,850,752; 3,939,350; 3,996,345; 4,277,437; 4,275,149; and 4,366,241.

The detectable moiety may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Means of detecting detectable moieties are well known to those of skill in the art. Thus, for example, where the detectable moiety is a radioactive label, means for detection include a scintillation counter or photographic film as in autoradiography. Where the detectable moiety is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDs) or photomultipliers and the like. Similarly, detectable enzymatic moieties may be detected by providing the appropriate substrates for the enzyme and detecting the resulting reaction product. Finally, simple colorimetric moieties may be detected simply by observing the color associated with the label. Thus, conjugated gold often appear pink, while various conjugated beads appear the color of the bead.

C. Capturing Biomolecules

Single step expression and capture of biomolecules is accomplished by housing one or more expression systems in a reaction space that is in fluid communication with a solid support having a surface capable of adsorbing at least one of the expressed biomolecules. By allowing the solid support to remain in fluid communication with the expression system, biomolecules produced by the expression system are contacted with the adsorbent surface(s) of the solid support immediately upon release from the production cell.

1. Solid Supports

Acceptable supports for use in the present invention can vary widely. A support can be porous or nonporous, but is preferably porous. It can be continuous or non-continuous, flexible or nonflexible. A support can be made of a variety of materials including supports made of ceramic, glassy, metallic, organic polymeric materials, or combinations thereof. Such supports can be magnetic, which allows for concentration and intensification of the signal.

Preferred supports include organic polymeric supports, such as particulate or beaded supports, woven and nonwoven webs (such as fibrous webs), microporous fibers, microporous membranes, hollow fibers or tubes. Polyacrylamide and mineral supports such as silicates and carbonates (e.g., hydroxyl apatite) can also be used. Woven and nonwoven webs may have either regular or irregular physical configurations of surfaces.

Porous materials are particularly desirable because they provide large surface areas. The porous support can be synthetic or natural, organic or inorganic. Suitable solids with a porous structure having pores of a diameter of at least about 1.0 nanometer (nm) and a pore volume of at least about 0.1 cubic centimeter/gram (cm³/g). Preferably, the pore diameter is at least about 30 nm because larger pores will be less restrictive to diffusion. Preferably, the pore volume is at least about 0.5 cm³/g for greater potential capacity due to greater surface area surrounding the pores. Preferred porous supports include particulate or beaded supports such as agarose, Styrofoam, and Sepharose.

For significant advantage, the supports are preferably hydrophilic, and have a high molecular weight (preferably, greater than about 5000, and more preferably, greater than about 40,000). Preferably, the hydrophilic polymers are water swellable to allow for greater infiltration of enzyme. Examples of such supports include cellulose, modified celluloses, agarose, polyvinyl alcohol (PVA), dextrans, amino-modified dextrans, polyacrylamide, modified guar gums, guar gums, xanthan gums, and locust bean gums and hydrogels.

The solid support may comprise any portion of the reaction vessel, typically the floor and/or walls. Alternatively, the solid support may be an inert material housed within the reaction space but otherwise independent of the reaction vessel, or may be housed outside, but in fluid communication with, the reaction space. In addition, more than one solid support may be in fluid communication with a common reaction space. This approach allows biomolecules to be contacted simultaneously with a plurality of different adsorbent surfaces, as illustrated in FIG. 3.

Typically, the solid support is adapted for use with the detectors employed in the methods of the present invention for detecting biomolecule(s) bound to and retained by the adsorbent. In one embodiment, the solid support is removably insertable into a desorption detector where an energy source can strike the spot and desorb the biomolecule. The solid support can be suitable for mounting in a horizontally and/or vertically translatable carriage that horizontally and/or vertically moves the solid support to successively position each predetermined addressable location of adsorbent in a path for interrogation by the energy source and detection of the biomolecule bound thereto. The solid support can be in the form of a conventional mass spectrometry chip.

Attachment of the adsorbent to the solid support can be accomplished through a variety of mechanisms. The solid support can be derivatized with a fully prepared adsorbent molecule by attaching the previously prepared adsorbent molecule to the solid support. Alternatively, the adsorbent can be formed on the solid support by attaching a precursor molecule to the solid support and subsequently adding additional precursor molecules to the growing chain bound to the solid support by the first precursor molecule. This mechanism of building the adsorbent on the solid support is particularly useful when the adsorbent is a polymer, particularly a biopolymer such as a DNA or RNA molecule. A biopolymer adsorbent can be provided by successively adding bases to a first base attached to the solid support using methods known in the art of oligonucleotide chip technology. See, e.g., U.S. Pat. No. 5,445,934 (Fodor et al.).

As few as two and as many as 10, 100, 1000, 10,000 or more adsorbents can be coupled to a single solid support. The size of the adsorbent site may be varied, depending on experimental design and purpose. However, it need not be larger than the diameter of the impinging energy source (e.g., laser spot diameter). The spots can continue the same or different adsorbents. In some cases, it is advantageous to provide the same adsorbent at multiple locations on the solid support to permit evaluation against a plurality of different eluants or so that the bound biomolecule can be preserved for future use or reference, perhaps in secondary processing. By providing a solid support with a plurality of different adsorbents, it is possible to utilize the plurality of binding characteristics provided by the combination of different adsorbents with respect to a single sample and thereby bind and detect a wider variety of different biomolecules. The use of a plurality of different adsorbents on a solid support for evaluation of a single sample is essentially equivalent to concurrently conducting multiple chromatographic experiments, each with a different chromatography column, but the present method has the advantage of requiring only a single system.

When the solid support includes a plurality of adsorbents, it is particularly useful to provide the adsorbents in predetermined addressable locations. By providing the adsorbents in predetermined addressable locations, it is possible to wash an adsorbent at a first predetermined addressable location with a first eluant and to wash an adsorbent at a second predetermined addressable location with a second eluant. In this manner, the binding characteristics of a single adsorbent for the biomolecule can be evaluated in the presence of multiple eluants that each selectively modifies the binding characteristics of the adsorbent in a different way. The addressable locations can be arranged in any pattern, but preferably in regular patters, such as lines, orthogonal arrays, or regular curves, such as circles. Similarly, when the solid support includes a plurality of different adsorbents, it is possible to evaluate a single eluant with respect to each different adsorbent in order to evaluate the binding characteristics of a given adsorbent in the presence of the eluant. It is also possible to evaluate the binding characteristics of different adsorbents in the presence of different eluants, or the affinity of different biomolecules for different binding partners, as described below.

a. Derivatizing Solid Supports

In order to be useful for the purposes of the invention, the support includes a reactive functional group that can be used to attach adsorbent materials or components to the support surface. Preferably, the reactive functional group is capable of undergoing rapid, direct, covalent coupling with the adsorbent materials to form a derivatized adsorbent surface. Preferably, the support includes at least one reactive functional group, such as a hydroxyl, carboxyl, sulfhydryl, or amino group that chemically binds to the enzyme substrate, optionally through a spacer group. Other suitable functional groups include N-hydroxysuccinimide esters, sulfonyl esters, iodoacetyl groups, aldehydes, epoxy, imidazolyl carbamates, and cyanogen bromide and other halogen-activated supports. Such functional groups can be provided to a support by a variety of known techniques. For example, a glass surface can be derivatized with aminopropyl triethoxysilane in a known manner.

A preferred embodiment of the invention comprises a solid support that is capable of functioning as an MS probe, preferably a SELDI MS probe. In one aspect of this preferred embodiment, the adsorbent is attached to a first solid support to provide a solid phase, such as a polymeric or glass bead, which is subsequently positioned on a second solid support which functions as the means for presenting the sample to the desorbing energy of the desorption detector. For example, the second solid support can be in the form a plate having a series of wells serving as reaction vessels located at predetermined addressable locations. One advantage of this embodiment is that the biomolecule can be adsorbed to the first solid support in one physical context, and transferred to a solid support that is a functional MS probe for analysis by desorption spectrometry.

b. Adsorbent Materials

Adsorbents are the materials that bind biomolecules expressed within or added to the reaction space of the present invention. Adsorbents used in the practice of the present invention are coupled to a solid support, frequently though a linker moiety as described above. Different adsorbents can exhibit grossly different binding characteristics, somewhat different binding characteristics, or subtly different binding characteristics. Adsorbents that exhibit grossly different binding characteristics typically differ in their bases of attraction or mode of interaction. The specific adsorbent(s) used in practicing the present invention is dependent upon the capture moiety possessed by the biomolecule to be bound and the nature of the expression system used. For example, if the capture moiety is an epitope tag, the adsorbent surface will contain a specific binding agent specifically recognizing the epitope tag. In cases where the biomolecule to be bound is secreted and the extracellular fluid of the expression system is substantially free of components that adsorb to the adsorbent surface, then the adsorbent surface may comprise any adsorbent material(s) binding the biomolecule. In the latter example, the adsorbent surface may be blocked after binding the biomolecule to prevent non-specific binding of molecules subsequently presented to the bound biomolecule. Blocking materials include serum albumins, casein, and other common proteins known in the art as suitable for this purpose. For further details regarding blocking strategies see Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series.

The adsorbent surface should display preferential binding of the expressed biomolecule(s) such that at least a partial purification can be achieved by binding the biomolecule(s) to the adsorbent surface with subsequent washing to remove unbound components of the expression system. Preferably binding between the adsorbent surface and the biomolecule(s) has specificity, and most preferably the binding is specific and has a dissociation constant of the order of 10⁻⁶, more preferably of the order of 10⁻⁹ or less.

The temperature at which the sample is contacted to the affinity molecule is a function of the particular sample and affinity molecule selected. Typically, contact is made under ambient temperature and pressure conditions, however, for some samples, modified temperature (typically 4° C. through 37° C.) and pressure conditions can be desirable and will be readily determinable by those skilled in the art.

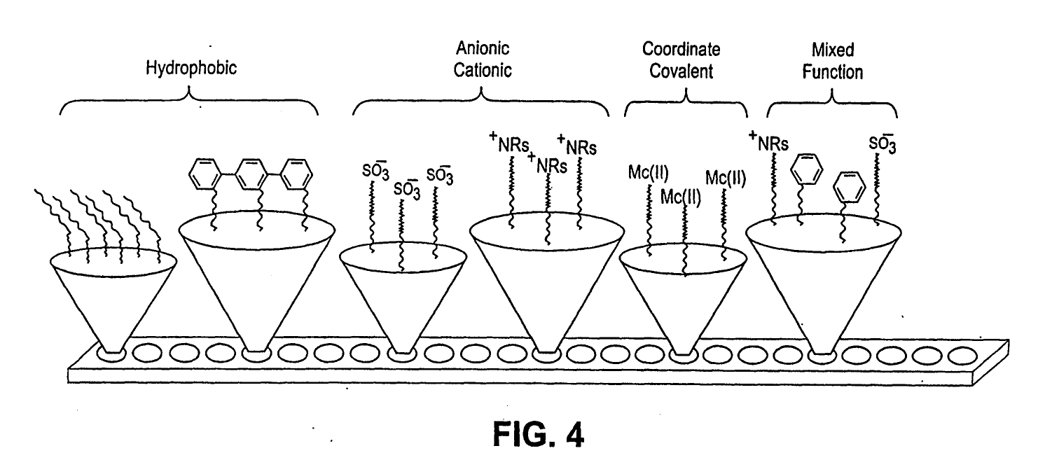

The basis of attraction between the adsorbent and the adsorbed biomolecule is generally a function of chemical or biological molecular recognition. Bases for attraction between an adsorbent and a biomolecule include, for example, (1) a salt-promoted interaction, e.g., hydrophobic interactions, thiophilic interactions, and immobilized dye interactions; (2) hydrogen bonding and/or van der Waals forces interactions and charge transfer interactions, such as in the case of a hydrophilic interactions; (3) electrostatic interactions, such as an ionic charge interaction, particularly positive or negative ionic charge interactions; (4) the ability of the biomolecule to form coordinate covalent bonds (i.e., coordination complex formation) with a metal ion on the adsorbent; (5) enzyme-active site binding; (6) reversible covalent interactions, for example, disulfide exchange interactions; (7) glycoprotein interactions; (8) biospecific interactions; or (9) combinations of two or more of the foregoing modes of interaction. That is, the adsorbent can exhibit two or more bases of attraction, and thus be known as a “mixed functionality” adsorbent. FIG. 4 depicts examples of some of these adsorbent chemistries.

Salt-Promoted Interaction Adsorbents

Adsorbents that are useful for observing salt-promoted interactions include hydrophobic interaction adsorbents. Examples of hydrophobic interaction adsorbents include matrices having aliphatic hydrocarbons, specifically C₁-C_{S} aliphatic hydrocarbons; and matrices having aromatic hydrocarbon functional groups such as phenyl groups. Hydrophobic interaction adsorbents bind biomolecules, which include uncharged solvent exposed amino acid residues, and specifically amino acid residues that are commonly referred to as nonpolar, aromatic and hydrophobic amino acid residues, such as phenylalanine and tryptophan. Specific examples of biomolecules that will bind to a hydrophobic interaction adsorbent include lysozyme and DNA. Without wishing to be bound by a particular theory, it is believed that DNA binds to hydrophobic interaction adsorbents by the aromatic nucleotides in DNA, specifically, the purine and pyrimidine groups.

Another adsorbent useful for observing salt-promoted interactions includes thiophilic interaction adsorbents, such as for example T-GEL™ which is one type of thiophilic adsorbent commercially available from Pierce, Rockford, Ill. Thiophilic interaction adsorbents bind, for example, immunoglobulins such as IgG. The mechanism of interaction between IgG and T-GEL™ is not completely known, but solvent exposed trap residues are suspected to play a role.

A third adsorbent, which involves salt-promoted ionic interactions and also hydrophobic interactions, includes immobilized dye interaction adsorbents. Immobilized dye interaction adsorbents include matrices of immobilized dyes such as for example CIBACHRON™ blue available from Pharmacia Biotech, Piscataway, N.J. Immobilized dye interaction adsorbents bind proteins and DNA generally. One specific example of a protein that binds to an immobilized dye interaction adsorbent is bovine serum albumin (BSA).

Hydrophilic Interaction Adsorbents

Adsorbents that are useful for observing hydrogen bonding and/or van der Waals forces based on hydrophilic interactions include surfaces comprising normal phase adsorbents such as silicon-oxide (i.e., glass). The normal phase or silicon-oxide surface, acts as a functional group. In addition, adsorbents comprising surfaces modified with hydrophilic polymers such as polyethylene glycol, dextran, agarose, or cellulose can also function as hydrophilic interaction adsorbents. Most proteins will bind hydrophilic interaction adsorbents because of a group or combination of amino acid residues (i.e., hydrophilic amino acid residues) that bind through hydrophilic interactions involving hydrogen bonding or van der Waals forces. Examples of proteins that will bind hydrophilic interaction adsorbents include myoglobin, insulin and cytochrome C.

In general, proteins with a high proportion of polar or charged amino acids will be retained on a hydrophilic surface. Alternatively, glycoproteins with surface exposed hydrophilic sugar moieties, also have high affinity for hydrophilic adsorbents.

Electrostatic Interaction Adsorbents

Adsorbents that are useful for observing electrostatic or ionic charge interactions include anionic adsorbents such as, for example, matrices of sulfate anions (i.e., SO₃⁻) and matrices of carboxylate anions (i.e., COO⁻) or phosphate anions (OPO₃⁻). Matrices having sulfate anions are permanent negatively charged. However, matrices having carboxylate anions have a negative charge only at a pH above their pKa. At a pH below the pKa, the matrices exhibit a substantially neutral charge. Suitable anionic adsorbents also include anionic adsorbents which are matrices having a combination of sulfate and carboxylate anions and phosphate anions. The combination provides an intensity of negative charge that can be continuously varied as a function of pH. These adsorbents attract and bind proteins and macromolecules having positive charges, such as for example ribonuclease and lactoferrin. Without wishing to be bound by a particular theory, it is believed that the electrostatic interaction between an adsorbent and positively charged amino acid residues including lysine residues, arginine residues, and histidyl residues are responsible for the binding interaction.

Other adsorbents that are useful for observing electrostatic or ionic charge interactions include cationic adsorbents. Specific examples of cationic adsorbents include matrices of secondary, tertiary or quaternary amines. Quaternary amines are permanently positively charged. However, secondary and tertiary amines have charges that are pH dependent. At a pH below the pKa, secondary and tertiary amines are positively charged, and at a pH above their pKa, they are negatively charged. Suitable cationic adsorbents also include cationic adsorbents which are matrices having combinations of different secondary, tertiary, and quaternary amines. The combination provides an intensity of positive charge that can be continuously varied as a function of pH. Cationic interaction adsorbents bind anionic sites on molecules including proteins having solvent exposed amino acid residues, such as aspartic acid and glutamic acid residues.

In the case of ionic interaction adsorbents (both anionic and cationic) it is often desirable to use a mixed mode ionic adsorbent containing both anions and cations. Such adsorbents provide a continuous buffering capacity as a function of pH. The continuous buffering capacity enables the exposure of a combination of biomolecules to eluants having differing buffering components especially in the pH range of from 2 to 11. This results in the generation of local pH environments on the adsorbent that are defined by immobilized titratable proton exchange groups. Such systems are equivalent to the solid phase separation technique known as chromatofocusing. Follicle stimulating hormone isoforms, which differ mainly in the charged carbohydrate components are separated on a chromatofocusing adsorbent.

Still other adsorbents that are useful for observing electrostatic interactions include dipole-dipole interaction adsorbents in which the interactions are electrostatic but no formal charge or titratable protein donor or acceptor is involved.

Coordinate Covalent Interaction Adsorbents

Adsorbents that are useful for observing the ability to form coordinate covalent bonds with metal ions include matrices bearing, for example, divalent and trivalent metal ions. Matrices of immobilized metal ion chelators provide immobilized synthetic organic molecules that have one or more electron donor groups that form the basis of coordinate covalent interactions with transition metal ions. The primary electron donor groups functioning as immobilized metal ion chelators include oxygen, nitrogen, and sulfur. The metal ions are bound to the immobilized metal ion chelators resulting in a metal ion complex having some number of remaining sites for interaction with electron donor groups on the biomolecule. Suitable metal ions include in general transition metal ions such as copper, nickel, cobalt, zinc, iron, and other metal ions such as aluminum and calcium. Without wishing to be bound by any particular theory, metals ions are believed to interact selectively with specific amino acid residues in peptides, proteins, or nucleic acids. Typically, the amino acid residues involved in such interactions include histidine residues, tyrosine residues, tryptophan residues, cysteine residues, and amino acid residues having oxygen groups such as aspartic acid and glutamic acid. For example, immobilized ferric ions interact with phosphoserine, phosphotyrosine, and phosphothreonine residues on proteins. Depending on the immobilized metal ion, only those proteins with sufficient local densities of the foregoing amino acid residues will be retained by the adsorbent. Some interactions between metal ions and proteins can be so strong that the protein cannot be severed from the complex by conventional means. Human P casein, which is highly phosphorylated, binds very strongly to immobilized Fe(III). Recombinant proteins that are expressed with a 6-Histidine tag, binds very strongly to immobilized Cu(II) and Ni(II).

Enzyme-Active Site Interaction Adsorbents

Adsorbents that are useful for observing enzyme-active site binding interactions include proteases (such as trypsin), phosphatases, kinases, and nucleases. The interaction is a sequence-specific interaction of the enzyme-binding site on the biomolecule (typically a biopolymer) with the catalytic binding site on the enzyme. Enzyme binding sites of this type include, for example, active sites of trypsin interacting with proteins and peptides having lysine-lysine or lysine-arginine pairs in their sequence. More specifically, soybean trypsin inhibitor interacts with and binds to an adsorbent of immobilized trypsin. Alternatively, serine proteases are selectively retained on immobilized L-arginine adsorbent and analogs such as p-aminobenzamidine.

Reversible Covalent Interaction Adsorbents

Adsorbents that are useful for observing reversible covalent interactions include disulfide exchange interaction adsorbents. Disulfide exchange interaction adsorbents include adsorbents comprising immobilized sulfhydryl groups, e.g., mercaptoethanol or immobilized dithiothrietol. The interaction is based upon the formation of covalent disulfide bonds between the adsorbent and solvent exposed cysteine residues on the biomolecule. Such adsorbents bind proteins or peptides having cysteine residues and nucleic acids including bases modified to contain reduced sulfur compounds. Another example of adsorbent useful for covalent reversible interaction comprises immobilized mercury atoms that form a covalent bond with exposed cysteine residues on the biomolecule.

Glycoprotein Interaction-Adsorbents

Adsorbents that are useful for observing glycoprotein interactions include glycoprotein interaction adsorbents such as adsorbents having immobilize lectins (i.e., proteins bearing oligosaccharides) therein, an example of which is concanavalin A, which is commercially available from Pharmacia Biotech of Piscataway, N.J. Such adsorbents function based on the interaction involving molecular recognition of carbohydrate moieties on macromolecules. Examples of biomolecules that interact with and bind to glycoprotein interaction adsorbents include glycoproteins, particularly histidine-rich glycoproteins, whole cells and isolated subcellular fractions.

Biospecific Interaction Adsorbents

Adsorbents that are useful for observing biospecific interactions are generically termed “biospecific affinity adsorbents.” Adsorption is considered biospecific if it is selective and the affinity (equilibrium dissociation constant, Kd) is at least 10⁻³ M to (e.g., 10⁻⁵ M, 10⁻⁷ M, 10⁻⁹ M). Examples of biospecific affinity adsorbents include any adsorbent that specifically interacts with and binds a particular biomolecule. Biospecific affinity adsorbents include for example, immobilized antibodies which bind to antigens; immobilized DNA which binds to DNA binding proteins, DNA, and RNA; immobilized substrates or inhibitors which bind to proteins and enzymes; immobilized drugs which bind to drug binding proteins; immobilized ligands which bind to receptors; immobilized receptors which bind to ligands; immobilized RNA which binds to DNA and RNA binding proteins; immobilized avidin or streptavidin which bind biotin and biotinylated molecules; immobilized phospholipid membranes and vesicles which bind lipid-binding proteins. Enzymes are useful adsorbents that can modify an biomolecule adsorbent thereto. Cells are useful as adsorbents. Their surfaces present complex binding characteristics. Adsorption to cells is useful for identifying, e.g., ligands or signal molecules that bind to surface receptors. Viruses or phage also are useful as adsorbents. Viruses frequently have ligands for cell surface receptors (e.g., gp120 for CD4). Also, in the form a phage display library, phage coat proteins act as agents for testing binding to targets. Biospecific interaction adsorbents rely on known specific interactions such as those described above. Other examples of biospecific interactions for which adsorbents can be utilized will be readily apparent to those skilled in the art and are contemplated by the present invention.

2. Biochips

Biochips are a preferred form of derivatized solid support for use in the present invention. This is because the biochip offer a means of concentrating biomolecules in a small area, can support a large range of adsorbent surfaces as described above, and are amenable to a variety of assay techniques, including fluorometric, colorimetric and mass spectroscopic, as discussed in greater detail below. Spatially, the biochip can form the floor of the reaction vessel or can be separate from the reaction vessel while in fluid contact with it.

Biochips may optionally be used with the present invention as convenient platforms for assaying samples produced by the invention. For example, FIG. 1 depicts a reaction vessel in the a chromatography column format. The reaction space within the column comprises a solid support and an expression system for one or more biomolecules. At least one of the biomolecules produced by the expression system comprises a capture moiety recognized by an adsorbent surface derivatized to the solid support. The biomolecule comprising the capture tag is adsorbed to the adsorbent surface. Additional molecular components may associate with the tagged biomolecule, including other biomolecules.

The captured biomolecule is then subjected to wash solutions comprising various amounts and combinations of eluants (washes A-D in the figure). Exemplary eluants include dissolved salts, glycerol, detergents, acids, bases, organics and the like. Each of these washes is collected and contacted to a biochip. The biochip comprises two or more adsorbent surfaces for adsorbing biomolecules. These adsorbent surfaces may specifically recognize particular proteins or capture moieties, or may comprise non-specific adsorbent surfaces, including those depicted in FIG. 4. Adsorbent surfaces compatible with use in a biochip format include those previously discussed for solid supports. A given biochip may comprise a single adsorbent surface, but more typically comprises multiple adsorbent surfaces, preferably arranged in a pattern of addressable locations suitable for automated analysis. More preferably, the adsorbent surfaces of the biochip are compatible with use in SELDI mass spectroscopy techniques, as the signal characteristics afforded by this method of analysis are superior to alternative biomolecular detection techniques.

The solid support portion of the biochip can be any solid material as described above, but preferably consists of materials compatible with use as an MS probe, more preferably a SELDI MS probe. The adsorbent surfaces are added to the chip by first derivatizing the solid support in at least two locations where an adsorbent surface can be coupled using a bifunctional linker. The linker includes at one end a functional group that can covalently bind with a functional group on the biochip surface, and a second functional group at another end for coupling to an absorbent material, as described herein. In addition to linkers previously described, aminopropyl triethoxysilane or aminoethyl disulfide can be used for this purpose, and are preferred for use on biochips.

III. Analyzing Captured Biomolecules

The methods and apparatus of the present invention provide a rapid means for detecting, isolating and purifying biomolecules, and analyzing the characteristics of biomolecular interactions. Through the use of the invention both the nature and the strength of these interactions between different biomolecules, ligands, and other compounds involved in or affecting biomolecular interactions, including small organic molecules, can be determined. Moreover, the present invention also provides a means for screening compounds that modulate enzyme activity.

Determinations of changes occurring between members of multisubunit complexes and receptor-ligand pairs are important because such changes are frequently associated with disease states. The present invention therefore provides a rapid and efficient means for diagnosing disease through characterization of binding interactions between biomolecules or between biomolecules and their ligands. In certain embodiments, the biomolecules of interest are interacting subunits of a common biomolecular complex. In other embodiments, the biomolecules are ligand receptor pairs. Still other embodiments comprise expressed biomolecules that are enzymes and/or one or more of their recognized substrates.

Once a biomolecule or complex has been captured, it is first subjected to a mild wash solution prior to any selective elution designed for analytical purposes. The mild wash solution is designed to remove contaminants frequently found in samples containing biomolecules. Typically an initial wash solution will be at a physiologic pH and ionic strength and the wash will be conducted under ambient conditions of temperature and pressure.

After the initial wash, the captured biomolecule or biomolecular complex can be analyzed in a variety of ways. For example, the captured molecule can be eluted from the capture adsorbent and further analyzed by testing its binding properties relative to other adsorbent surfaces. Molecular complexes can be subjected to fractional elution analysis to determine the conditions required to cause the complex, or complex components, to release from their binding partners or the adsorbent surface itself. The particular composition of the mild wash solution will be dependent upon the nature of the biomolecule of interest. Formulation of suitable mild wash solutions can be performed by one of skill in the art without undue experimentation. Methods for removing contaminants, including low stringency washing methods, are available in published form, for example in Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series.

Another alternative are tests for enzymatic activity. These tests can be conducted in one of two modes, either by expressing and capturing the enzyme and then contacting the enzyme with putative substrates, or by expressing and capturing the substrate and contacting the captured substrate with putative enzymes that could recognize it. Still another aspect is testing enzymes with putative substrates and then capturing resulting products. This last variation is a useful variation for analyzing enzyme activities that add a distinctive chemical group to molecules, where the distinctive chemical group can serve as a capture moiety. One example of an enzyme system conducive to study using this latter variation are protein kinases.

Under certain circumstances, e.g., when the components of the biomolecular complex of interest are still immobilized after an elution wash that is of a particular stringency has been carried out, then the stringent wash may be the only wash step performed. When it is anticipated that the biomolecular complex components of interest will remain associated with the adsorbent surface of the solid support after the most stringent wash, then the solid support used is preferably an MS probe, most preferably a SELDI MS probe, with an adsorbent surface capable of specifically binding the biomolecular complex.

Any detection method or device compatible with the assay system and the nature of the biomolecule of interest may be used in practicing the present invention. Spectroscopic detectors rely on a change in refractive index; ultraviolet and/or visible light absorption, or fluorescence after excitation with a suitable wavelength to detect reaction components. Exemplary detection methods include fluorimetry, absorbance, reflectance, and transmittance spectroscopy. Changes in birefringence, refractive index, or diffraction may also be used to monitor complex formation or reaction progression. Particularly useful techniques for detecting molecular interactions include surface plasmon resonance, ellipsometry, resonant mirror techniques, grating-coupled waveguide techniques, and multi-polar resonance spectroscopy. These techniques and others are well known and can readily be applied to the present invention by one skilled in the art, without undue experimentation. Many of these methods and others may be found, for example, in “Spectrochemical Analysis” Ingle, J. D. and Crouch, S. R., Prentice Hall Publ. (1988) and “Analytical Chemistry” Vol. 72, No. 17.

A preferred method of detection involves SELDI mass spectroscopy, as discussed below and in U.S. Pat. No. 6,225,047 B1, which is hereby incorporated by reference.

A. Analysis Methodologies for Captured Expression Products

Each of the analytical approaches discussed below involves in situ expression and capture of biomolecules using the expression systems and the adsorbent surfaces of the present invention.

1. Express-Capture-Wash-Detect

In one embodiment, a biomolecule expressed and captured in situ using the techniques of the present invention can be directly detected using one of the detection strategies described herein. In this embodiment, the expressed biomolecule is captured to an adsorbent surface recognizing the capture moiety of the biomolecule. The captured biomolecule is typically subjected to a mild buffer wash, as described above, to remove uncaptured or adventitiously bound components of the system. The captured biomolecule can then be readily detected using one of the detection approached discussed herein. As noted above, a preferred method of detection of the present invention is mass spectroscopy, as this method allows detection of multiple components of a system regardless of composition, provided each component has a molecular weight distinct from other components of the system. A particularly preferred method is SELDI mass spectroscopy, which allows analysis of a sample directly from an adsorbent surface without the addition of matrix materials that can degrade signal to noise. As described herein, certain embodiments of the present invention provide an adsorbent surface that is part of a biochip capable of functioning as an MS probe.

Other embodiments provide adsorbent surfaces that are part of a functionalized solid support other than a biochip, for example a porous matrix material or plastic surface. These latter supports are not suitable for mass spectroscopy use, but biomolecules that are to be analyzed may be eluted from these functionalized solid supports and transferred to MS probes for analysis. Any suitable eluant may be used for this purpose, including denaturing agents such as chaotropes and organic solvents, provided that the elution agent does not interfere with mass spectroscopic analysis. One type of elution agent for the present invention is the capture moiety or a molecular agent comprising the capture moiety of the biomolecule adsorbed to the adsorbent surface. By contacting the adsorbed biomolecule with an excess of capture moiety, the biomolecule can be freed from the surface through a competitive mechanism.

Biomolecules bound on the adsorbent surface of the MS probe can be desorbed and ionized using mass spectrometry. Any suitable ionizing mass spectrometer, e.g., a gas phase ion spectrometer, can be used. In a typical mass spectrometer, an MS probe carrying the biomolecule bound by the adsorbent is introduced into an inlet system of the mass spectrometer. The biomolecule is then desorbed by a desorption source such as a laser, fast atom bombardment, high-energy plasma, electrospray ionization, thermospray ionization, liquid secondary ion MS, field desorption, etc. The generated desorbed, volatilized species consist of preformed ions or neutrals which are ionized as a direct consequence of the desorption event. An ion optic assembly collects generated ions, and then a mass analyzer disperses and analyzes the passing ions. A suitable detector detects the ions exiting the mass analyzer. The detector then translates information of the detected ions into mass-to-charge ratios. Detection of the presence of a biomolecule will typically involve detection of signal intensity. Any of the parts of a mass spectrometer (e.g., a desorption source, a mass analyzer, a detector, etc.) can be combined with other suitable parts described herein or others known in the art in embodiments of the invention.

Preferably, a laser desorption time-of-flight mass spectrometer is used in embodiments of the invention. In laser desorption mass spectrometry, a probe adsorbent comprising a biomolecule is introduced into an inlet system. The pathway components are desorbed and ionized into the gas phase by laser from the ionization source. An ion optic assembly collects the ions generated, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of pathway components of specific mass to charge ratio.

Data generated by desorption and detection of biomolecules can be analyzed using any suitable means. In one embodiment, data is analyzed with the use of a programmable digital computer. The computer program generally contains a readable medium that stores codes. Certain code can be devoted to memory that includes the location of each feature on a probe, the identity of the capture reagents at that feature and the elution conditions used to wash the adsorbent surface. The computer also contains code that receives as input, data on the strength of the signal at various molecular masses received from a particular addressable location on the probe. This data can indicate the number of biomolecules detected, including the strength of the signal generated by each biomolecule.

Data analysis can include the steps of determining signal strength (e.g., height of peaks) of biomolecules detected and removing “outerliers” (data deviating from a predetermined statistical distribution). The observed peaks can be normalized, a process whereby the height of each peak relative to some reference is calculated. For example, a reference can be background noise generated by instrument and chemicals (e.g., MS matrix), which is set as zero in the scale. Then the signal strength detected for each biomolecule or biomolecular complex can be displayed in the form of relative intensities in the scale desired (e.g., 100). Alternatively, a standard may be admitted with the sample so that a peak from the standard can be used as a reference to calculate relative intensities of the signals observed for each multicomponent biological complex component detected.

The computer can transform the resulting data into various formats for displaying. In one format, referred to as “spectrum view or retentate map,” a standard spectral view can be displayed, wherein the view depicts the quantity of biomolecules reaching the detector at each particular molecular weight. In another format, referred to as “peak map,” only the peak height and mass information are retained from the spectrum view, yielding a cleaner image and enabling biomolecules with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting biomolecules that are up- or down-regulated compared to control. Profiles (spectra) from any two samples may be compared visually. In yet another format, Spotfire Scatter Plot can be used, wherein components that are detected are plotted as a dot in a plot, wherein one axis of the plot represents the apparent molecular weight of the multicomponent biological complex components detected and another axis represents the signal intensity of components detected. For each sample, biomolecules that are detected and the amount of components present in the sample can be saved in a computer readable medium. This data can then be compared to a control (e.g., a profile or quantity of components detected in control, e.g., from healthy subjects).

Data generated by desorption and detection of biomolecules in a test sample can be compared to a control data to determine, for example, effects of biomolecular modulators. Control data refers to data obtained from comparable samples from a normal cell or person, which or who is known to have no defects in the biomolecules, or data obtained in the absence of activity or interaction modulators. For each biomolecule being analyzed, a control amount of the same biomolecule from a normal sample or the absence of modulators is determined. Preferably, the control amount of each biomolecule is determined based upon a significant number of samples.

If the test amount of particular biomolecules is significantly increased or decreased compared to the control amount of the biomolecule, then this is a positive indication that the modulator has affected the test sample. For example, if the test amount of a biomolecule (or biomolecular activity) is increased or decreased by at least 1.5 fold, 2 fold, 5 fold or 10 fold compared to the control amount, then this is an indication that the test sample is susceptible to modulation by the modulator being tested.

2. Express-Capture-Wash-Elute

In another embodiment, the present invention provides for a method of isolating a biomolecule expressed in situ comprising capture of the biomolecule to an adsorbent surface; washing away uncaptured components; and eluting the biomolecule from the adsorbent surface. The biomolecule can then be readily detected using a detection method described herein.

Captured biomolecules and biomolecular complexes can be washed with any number of wash solutions of differing elution stringencies for subsequent, independent multi-dimensional analysis, depending on the application requirements. These wash steps may be preceded by an initial mile wash to remove unbound or weakly bound material present in the biosample comprising the biomolecules to be tested, as described above. Typically, to provide a multi-dimensional analysis, each adsorbent surface of a solid support is washed with at least an initial mild wash solution to remove contaminants from the sample and one wash solution of greater stringency. Washing the biomolecular complex in this way typically modifies the complex component population retained on a specified adsorbent surface. The combination of the binding characteristics of the component of the complex and the elution characteristics of the wash solutions provide the selectivity conditions that control which components are retained on the solid support bound to the adsorbent surface. Thus, the washing step selectively removes components from the biomolecular complex.

Washing an adsorbent surface having the biomolecular complex bound thereto can be accomplished by bathing, soaking, or dipping the solid support having the adsorbent surface and biomolecular complex bound thereon in an wash; or by rinsing, spraying, or washing over the solid support with the wash.

The foregoing method is also useful when adsorbent surfaces are provided at a plurality of predetermined addressable locations, whether the adsorbent surfaces are all the same or different. However, when the biomolecular complex is bound to adsorbent surfaces at a plurality of locations, the washing step may alternatively be carried out using a more systematic and efficient approach. FIG. 4 is a schematic representation of a chip for this purpose. The step of washing can be carried out by washing an adsorbent surface at a first location with one wash, then washing a second adsorbent surface with another wash, then desorbing and detecting the biomolecular complex components retained by the first adsorbent surface and thereafter desorbing and detecting biomolecular complex retained by the second adsorbent surface. In other words, all of the adsorbent surfaces exposed to the initial wash together and thereafter biomolecular complex components released from each adsorbent surface location can be individually analyzed. If desired, after detection of the biomolecular complex components released from each adsorbent surface location, a second stage of elution washes for each adsorbent surface location may be conducted followed by a second stage of detection and/or analysis. The steps of washing all adsorbent surface locations, followed by desorption and detection of released components for each adsorbent surface location can be repeated for a plurality of different elution washes. In this manner, and entire array may be utilized to determine efficiently the character of biomolecular complexes in a sample.

To increase the wash stringency of a wash solution, buffers and other additives can be incorporated into the wash solution. Additives include, but are not limited to, ionic interaction modifier (both ionic strength and pH), water structure modifier, hydrophobic interaction modifier, chaotropic reagents, and affinity interaction displacers. Specific examples of these additives can be found in, e.g., PCT publication WO98/59360 (Hutchens and Yip). The selection of a particular wash solution or additive is dependent on experimental conditions (e.g., types of affinity molecules used or biomolecular complex to be detected), and can be determined by those of skill in the art.

Charge-Based Washes

Washes that modify the selectivity of the affinity molecule based upon charge include known pH buffers, acidic solutions, and basic solutions. By washing the biomolecular complex bound to a given affinity molecule with a particular pH buffer, the strength of the bond between the affinity molecule and the biomolecular complex in the presence of the particular pH buffer can be challenged. Those biomolecular complexes that are less competitive than wash components for the affinity molecule at the pH of the wash will be desorbed from the affinity molecule and eluted, leaving bound only those biomolecular complexes that bind more strongly to the affinity molecule at the pH of the wash.

Ionic Strength-Based Washes

Washes that modify the selectivity of the affinity molecule with respect to ionic strength include salt solutions of various concentrations. The amount of salt solublized in the wash solution affects the ionic strength of the wash and modifies the affinity molecule binding ability correspondingly. Washes containing a low concentration of salt provide a slight modification of the affinity molecule binding ability with respect to ionic strength. Washes containing a high concentration of salt provide a greater modification of the affinity molecule binding ability with respect to ionic strength.

Water Structure-Based Washes

Washes that modify the selectivity of the affinity molecule with respect to water structure include urea or chaotropic salt solutions. Typically, urea solutions include, e.g., solutions ranging in concentration from 0.1 to 8 M. Chaotropic salts which can be used to provide washes include sodium thiocyanate. Water structure-based washes modify the ability of the affinity molecule to bind the biomolecular complex in the presence of glycerol, ethylene glycol, and organic solvents.

Detergent-Based Washes

Washes that modify the selectivity of the affinity molecule with respect to surface tension and biomolecular complex structure include detergents and surfactants. Suitable detergents for use as washes include ionic and nonionic detergents. Detergent-based washes modify the ability of the affinity molecule to bind the biomolecular complex as the surface tension between the biomolecular complex and affinity molecule is modified. Hydrophobic interactions are modified and charge groups are introduced, e.g., ionic detergents such as SDS.

Hydrophobicity-Based Washes

Washes that modify the selectivity of the affinity molecule with respect to dielectric constant are those washes that modify the selectivity of the affinity molecule with respect to hydrophobic interaction. Examples of suitable washes that function in this capacity include ethylene glycol, and organic solvents such as propanol, acetonitrile, and glycerol, and detergents such as CHAPS, TWEEN, and NP-40.

Combinations of Washes

Suitable washes can be selected from any of the foregoing categories or can be combinations of two or more of the foregoing washes. Washes that comprise two or more of the foregoing washes are capable of modifying the selectivity of the affinity molecule for the biomolecular complex based on multiple elution characteristics.

3. Express-Capture/Interact-Detect

a. Identifying Binding Partners

A third embodiment of the invention involves analyzing the interaction of biomolecules expressed and captured as described herein with other biomolecules through intermolecular binding interactions. To characterize biomolecular-binding interactions, at least one of the component biomolecules is immobilized to a solid support by adsorbing it to an adsorbent surface. As described previously, this is accomplished by selecting an adsorbent material that recognizes and binds a capture moiety of at least one biomolecule of interest (or vice versa).

In addition to the at least one molecule to be bound to the solid support, other components of the complex under study are also included in the reaction vessel. Complex components may be introduced in any order compatible with the application, thereby allowing adsorption of the tagged molecule before, after, or during an intermediate stage of complex formation. Inclusion of the complex components may be through expression systems of the invention located within the reaction space, or may be mixed with the contents of the reaction space, provided at least one component of the complex is introduced by production of the component through the action of an expression system while residing within the reaction space. Preferably all components of the complex are introduced via in situ expression.

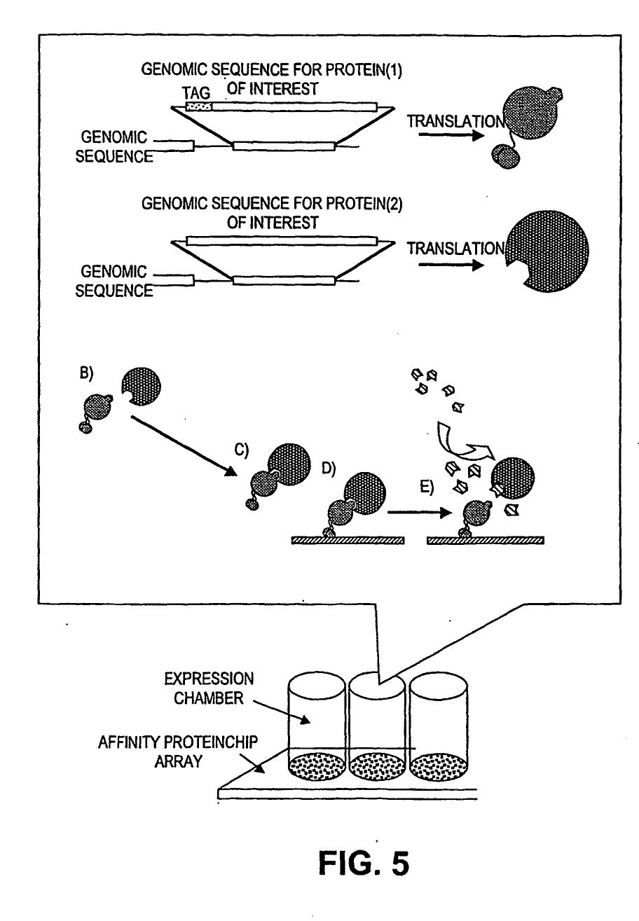

FIG. 5 schematically depicts the embodiment described above. As depicted, an expression system occupies a reaction space directly above an adsorbent surface positioned at an addressable location on the surface of a biochip; the biochip providing the solid support. The illustration depicts the expression system producing a biomolecule tagged with a capture moiety, and a second biomolecule that is a binding partner of the tagged biomolecule (“B)” in the figure). Both biomolecules are expressed at the same time, allowing them to interact prior to association of the capture moiety with the adsorbent surface. As the adsorbent surface is in fluid communication with the reaction space, the tagged biomolecule is free to diffuse to, and be captured by, the adsorbent surface.

An alternative approach provides multiple putative binding partners for the captured molecule. Again, putative binding partners may be added to the reaction space from an exogenous source or expressed in situ by an expression system of the present invention. Once contacted to the captured biomolecule putative binding partners are provided sufficient conditions to allow any molecule with the inherent capacity to bind to the captured biomolecule to do so. Any nascent complexes formed are then washed with a mild wash solution to remove unbound or adventitiously bound material. The resulting complexes are then detected with a suitable detection system as described herein.

As one of ordinary skill in the art will appreciate, these methods lend themselves to competitive binding studies to determine the relative affinities of different binding partners. Briefly, competitive binding studies are performed by varying the concentration of one binding partner relative to another and tracking the relative amount of each (or either) binding partner associated with the captured biomolecule or biomolecular complex.

Biomolecular complex interactions may also be studied using selective washing of the captured complex with wash solutions of differing stringencies, as described above. Each of the fractionating washes described possesses a different capability of disrupting intermolecular interactions, and many differ in the mechanism of disruption. As will be appreciated by one of ordinary skill in the art, these differences in properties are characteristics that can be utilized to determine the nature of the biomolecular interactions maintaining complexes of biomolecules. As an example, alterations in wash solution hydrophobicity will effect hydrophobic interactions. Alterations in ionic strength with predominantly effect ionic interactions, and so forth. Moreover, a correlation between the degree of change necessary to disrupt a molecular interaction and the strength of the molecular interaction is also typically found.

As described previously, biomolecules may be detected by any of the known methods including the preferred detection method, SELDI MS.

Other assays may be used to search for agents that bind to captured biomolecules of the present invention. One such screening method to identify direct binding of test ligands to a biomolecule is described in U.S. Pat. No. 5,585,277, incorporated herein by reference. This method relies on the principle that proteins generally exist as a mixture of folded and unfolded states, and continually alternate between the two states. When a test ligand binds to the folded form of a biomolecule (i.e., when the test ligand is a ligand of the biomolecule), the biomolecule molecule bound by the ligand remains in its folded state. Thus, the folded biomolecule is present to a greater extent in the presence of a test ligand that binds the biomolecule, than in the absence of a ligand. Binding of the ligand to the biomolecule can be determined by any method that distinguishes between the folded and unfolded states of the biomolecule. The function of the biomolecule need not be known in order for this assay to be performed. Virtually any agent can be assessed by this method as a test ligand, including, but not limited to, polypeptides, proteins, lipids, polysaccharides, polynucleotides small organic molecules, and metals.

b. Detect Enzymatic Activity

The present invention provides methods for determining enzyme activities of captured molecules and screening combinatorial libraries for putative substrates. Enzymes can include kinases, phosphatases, glycosidases, deglycosidases, proteases, etc. Briefly, expressed enzymes are tagged and captured to an adsorbent surface, as described above. The captured enzyme is then contacted with a substrate or panel of potential substrates, creating a reaction solution. The substrate(s) can be added as a unique component to the reaction space, or more preferably introduced via in situ expression. After sufficient time has elapsed for the reaction to proceed, the reaction solution is sampled and assayed to determine the presence of and/or amount of substrate consumed and/or product produced. The substrate and/or product may contain a second unique affinity tag for capture onto a second adsorbent surface, as described above.

In another embodiment the enzyme and a panel of substrates can be included in the reaction space without an affinity tag. The enzyme and/or substrates can be added as a unique component to the reaction space, or more preferably introduced via in situ expression. After the enzyme contacts the substrate(s) and a sufficient time has elapsed for the reaction to proceed, the substrate will be converted into product containing an affinity tag. This affinity tag can then be captured by the adsorbent surface. Such an example involves kinases whereby when the kinase is brought in contact with an un-phosphorylated substrate and becomes phosphorylated. The phosphorylated substrate (product) has affinity to the adsorbent surface, such as an IMAC surface.

In yet another aspect, the substrate or substrate library can be expressed in the reaction space of the invention, with each substrate comprising a capture moiety. In this aspect, the enzyme can be added exogenously, or can be expressed in situ. In this aspect, the substrate/product is captured to an adsorbent surface of the invention after reaction with the enzyme. The captured substrate/product can be optionally washed to remove unbound material and assayed to determine the extent of the reaction.

Using these techniques a panel of enzymes can be screened with a known substrate to identify enzymes that recognize and convert the substrate to product. Alternatively, a known enzyme activity can be captured to the adsorbent surface and screened using a panel of putative substrates to determine which members of the panel are indeed substrates of the captured enzyme.

A variety of methods are available for detecting the presence of reactants and products in a solution, the particular method employed being dependent upon the nature of the molecular species to be detected. Exemplary detection methods for enzyme assays include various forms of spectroscopy including fluorescence, absorbance, reflectance, transmittance, and birefringence. Alterations in refractive index and diffraction of the reaction solution can also be determinative, when these characteristics are modified by progression of the chemical reaction catalyzed by the enzyme under study.

Assay techniques for detecting enzyme activity are available widely available in the prior art for example the series “Methods in Enzymology,” published by Academic Press, consists now of over 350 volumes relating to enzyme assay techniques and theory dating back over 40 years.

B. Interaction Modulator Assays

Still other aspects of the present invention are screening assays for the detection of interaction modulators and modulators of enzyme activity. These methods typically involve carrying out the interaction and enzyme activity assays noted above in the presence of one or more potential modulators. The extent of interaction or the enzyme activity is then determined and compared against control experiments conducted in the absence of modulator(s). Potential modulators include biomolecules, small chemical compounds, a biological entity, such as a protein, sugar, nucleic acid or lipid, or inorganic salts or metals. Typically, test compounds will be small chemical molecules and peptides.

1. Interaction Modulators

To perform modulator assays, it is desirable to immobilize the biomolecule of interest to a solid support by capture with an adsorbent surface. Briefly, the methodology involves binding a biomolecule or molecular complex to an adsorbent surface, as described above. The captured biomolecule can then be optionally washed to remove unbound material. The captured biomolecule or complex is then contacted with a candidate compound and the effect(s) on the biomolecule or complex detected. In the context of this embodiment, a “biomolecule” includes bacteriophage, virus particles, and even whole cells that express a biomolecule comprising a capture moiety recognized and adsorbed by an adsorbent material of the invention. Thus an expression system comprising a membrane protein having a capture moiety is a biomolecule that can be adsorbed to an adsorbent surface of the invention and used as a tool for screening candidate compounds, as described herein.

Candidate compounds can be any small chemical compound, or a biological entity, such as a protein, sugar, nucleic acid or lipid. Typically, test compounds will be small chemical molecules and peptides. The assays are designed to screen large chemical libraries by automating the assay steps and providing compounds from any convenient source to assays, which are typically run in parallel (e.g., in microtiter formats on microtiter plates or similar formats, as depicted in FIG. 5, in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptides (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-921S (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Another approach uses recombinant bacteriophage to produce large libraries. Using the “phage method” (Scott and Smith, Science 249:386-390, 1990; Cwirla, et al, Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

A number of well-known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

2. Detection of Enzyme Modulators

The present invention also provides high-throughput screening for modulators of known enzyme activities. Screening for modulators is carried out by conducting high-throughput enzyme assays of known enzymes using known substrates in the presence and absence of a putative modulator compound. Briefly, an exemplary method involves tagging a known enzyme with a capture moiety. The enzyme is then immobilized to a solid support using an adsorbent surface recognizing the capture moiety, as described above, and can be optionally washed to remove unbound material. A known substrate for the enzyme and the putative modulator of enzyme activity is then contacted to the enzyme. Typically, the putative modulator will be placed in contact with the enzyme prior to addition of the substrate, but this is not always the case and the invention contemplates applications where the substrate would be added first, followed by addition of the inhibitor. Reactions may also require co-factors, metal ions and the like, necessary for the enzyme reaction to proceed. Addition of such ancillary components or other modulators or modifiers can be in any order and is generally dependent upon the application being pursued.

Alterations in enzymatic rate are then determined by analyzing the amount of product produced or substrate converted at one or more time points after the enzymatic reaction is commenced. Methods for determining reaction rates for enzymes reactions are well known in the art and may be found in reference texts such as the “Methods in Enzymology” series mentioned above.

Putative modulators can be derived from biological sources, mined from non-biological sources, or synthesized. Methods for making combinatorial libraries of putative modulator proteins are well known and are discussed supra.

IV. Kits

Another aspect of the invention provides kits comprising an apparatus for expression and capture of biomolecules that has at least one reaction vessel containing an expression system for at least one biomolecule tagged with a capture moiety, and a solid support having an adsorbent surface that binds the tagged biomolecule. One step expression and capture of biomolecules is accomplished when using the device as the solid support is in fluid communication with the expression system. Some kit embodiments also include instruction materials for using the apparatus. Still other kits further comprise expression system(s) for producing biomolecules with capture moieties recognized by at least one of the adsorbent surfaces included in the kit.

Alternative kits comprise MS probes optionally including different adsorbent surface chemistries. The kits of the invention have many applications. For example, the kits can be used to determine binding affinities, screen combinatorial libraries for binding ligands, activity modulators and other drug candidates.

In some embodiments, the kit may comprise an eluant (as an alternative or in combination with instruction material) for washing the adsorbent, which eluant allows retention of biomolecular components when washed with eluant. Alternatively or additionally, the kit may further comprise an instruction material for washing the adsorbent with the eluant after contacting the adsorbent with a sample. Such kits can be prepared from the materials described above, and the previous discussion of these materials (e.g. probe adsorbents, expression systems, washing solutions, etc.) is fully applicable to this section and will not be repeated.

Optionally, the kit may further comprise standard or control information so that the test sample can be compared with the control information standard to determine if the activities being studied in a test sample are normal and/or the assay for these activities is functioning properly. For example, standards include samples of biomolecules of known activities or binding affinities free from contaminating activities and binding partners.

II. Development of Classification Models

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of target analytes can be analyzed with the use of a programmable digital computer. The computer program analyzes the data to indicate the number of proteins detected, and optionally the strength of the signal and the determined molecular mass for each target analyte detected. Data analysis can include steps of determining signal strength of a target analyte and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a target analyte according to the present invention. The software also can subject the data regarding observed target analyte peaks to classification tree or ANN analysis, to determine whether a target analyte peak or combination of target analyte peaks is present that can classify a sample as belonging to one of two or more different groups. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

While single target analytes have traditionally been used to distinguish samples, such as presence or absence of disease, scientists and physicians have taken increasing interest in the use of multiple makers. This approach has become possible as a result of new technologies, such as gene arrays and affinity mass spectrometry, that allow differential detection of many different molecules in a clinical sample. The discovery of patterns of molecules that can be correlated with a clinical parameter involves the multivariate analysis of measurements of a plurality of molecules, such as proteins, in a sample.

Accordingly, in one aspect this invention provides a method for discovering patterns of proteins expressed in a sample, which patterns correlate with a phenotypic or physiological parameter of interest. This method involves training a learning algorithm with a learning set of data that includes measurements of the aforementioned molecules and generating a classification algorithm that can classify an unknown sample into a class represented by clinical parameter.

The method involves, first, providing a learning set of data. The learning set includes data objects. Each data object represents a sample for which expression data has been developed. The expression data included in the data object includes the specific measurements of polypeptides expressed in a sample and captured according to the methods of this invention. Each sample is classified into one of at least two different classes. For example, the clinical parameters could include presence or absence of an expression vector in the expression system.

In a preferred embodiment, the learning set will be in the form of a table in which, for example, each row is data object representing a sample. The columns contain information identifying the subject, data providing the specific measurements of each of the molecules measured and optionally identifying the clinical parameter associated with the subject.

The learning set is then used to train a classification algorithm. Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, each data object includes data indicating the clinical parameter class to which the subject belongs. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines). A preferred supervised classification method is a recursive partitioning process. Recursive partitioning processes use recursive partitioning trees to classify spectra derived from unknown samples.

In other embodiments, the classification models that are created can be formed using unsupervised learning methods. Unsupervised classification attempts to learn classifications based on similarities in the training data set. In this case, the data representing the class to which the subject belongs is not included in the data object representing that subject, or such data is not used in the analysis. Unsupervised learning methods include cluster analyses. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application 2002 0193950 A1 (Gavin et al., “Method or analyzing mass spectra”), U.S. Patent Application 2003 0004402 A1 (Hitt et al., “Process for discriminating between biological states based on hidden patterns from biological data”), and U.S. Patent Application 2003 0055615 A1 (Zhang and Zhang, “Systems and methods for processing biological expression data”).

Thus trained, learning algorithm will generate a classification model that classifies a sample into one of the classification groups. The classification model usually involves a subset of all the markers included in the learning set. The classification model can be used to classify an unkown sample into one of the groups.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

### Example 1

### On-Chip Monitoring of Recombinant His-PelleC Protein Expression in E. coli by SELDI

Cell Culture and Expression System

IPTG inducible vector pQE30 (Qiagen) was used to clone the His-PelleC gene. The protein was expressed in E. coli. M15 cells. An overnight culture of E. coli cells containing the expression plasmid of interest was grown up in 5 ml of LB/antibiotics at 37° C. The culture was diluted 1:10 in fresh LB/antibiotics before loading onto chips.

Preparation of IMAC-3-Ni Chips and On-Chip Growth

IMAC-3 chips were charged with 5 ul of 50 mM nickel chloride for 5 min each, twice, followed by rinsing with 5 ul of water once and 5 ul of PBS twice for 5 min each. Load the chips in a 96-well format bio-processor. In each well, aliquot diluted cell culture in a volume varying from 10 ul to 125 ul. The cells were grown for 1.5 hrs at 37° C. until OD₆₀₀ reaches 0.6. IPTG was added to a final concentration between 0 to 1 mM. The cells were grown for an additional 1-3 hrs at 37° C. to allow protein expression. A schematic diagram is outlined in FIG. 1.

Lysis of Cells and Binding of His-Tagged Protein to IMAC-Ni Surface

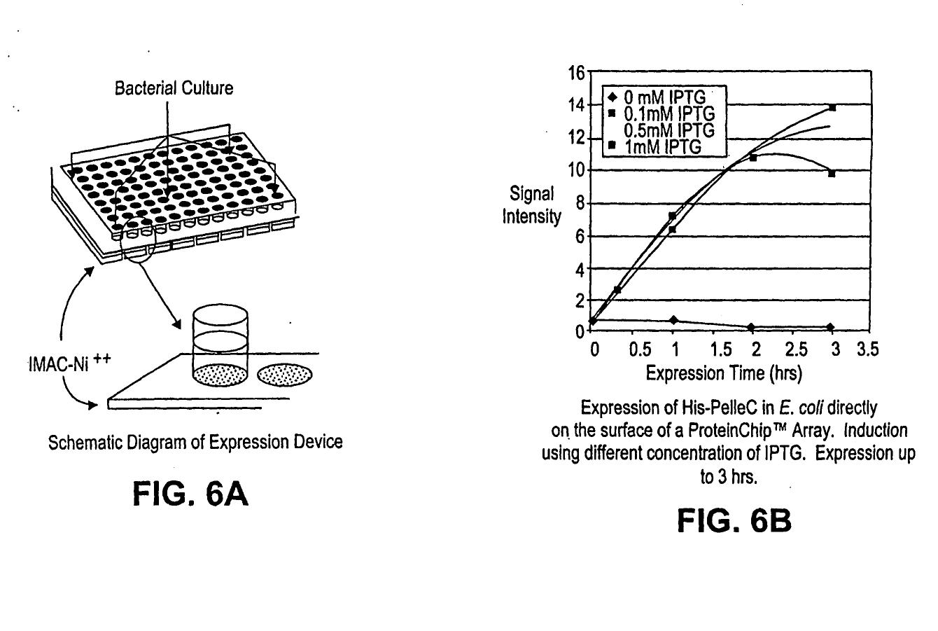

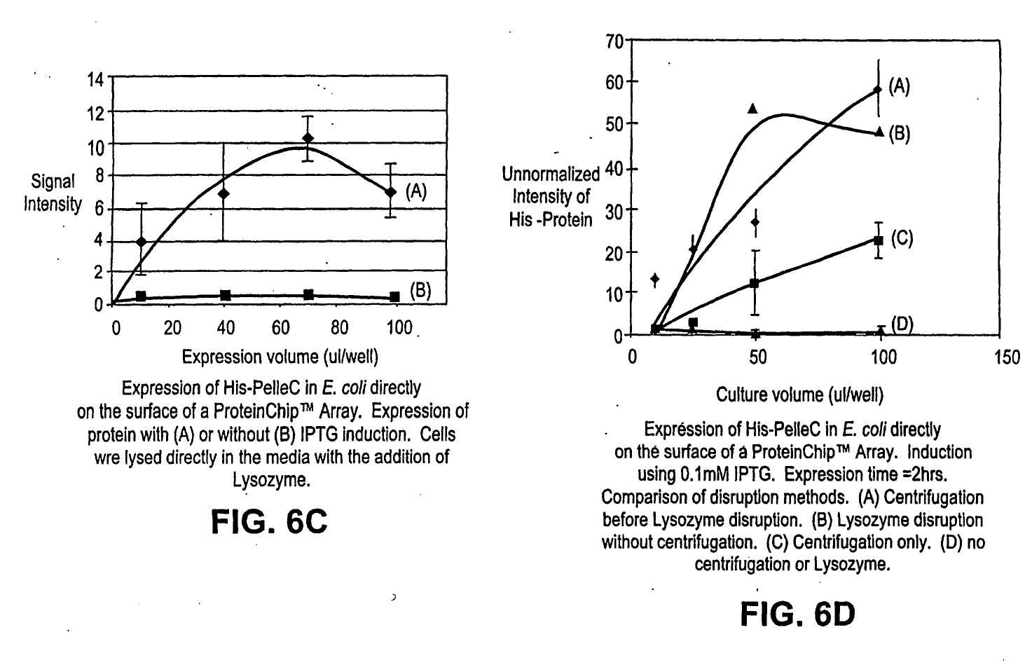

At the end of induction, the cells were spun down in the bio-processor at 2,000 rpm for 10 min at 4° C. and medium was removed from each well. Ten micro liters of a buffer containing 50 mM NaH₂PO₄, 300 mM NaCl, 10 mM imidazole and 1 mg/ml lysozyme, pH 8 was added to each well. The cells were lysed for 30 min before 90 ul of the buffer (50 mM NaH₂PO₄, 300 mM NaCl, 10 mM imidazole) was added to dilute out the lysozyme. Continue incubation for 1 hr to allow binding of His-tagged protein to the surface (FIG. 6).

Alternatively, at the end of induction, the cells were not centrifuged, instead a 10× solution containing 10 mg/ml lysozyme was added directly to the medium in the wells to lyse the cells for 30 min. Continue to incubate in the same medium for 1 hr.

At the end of 1 hr, the solution was removed, and 100 ul of a buffer containing 50 mM NaH₂PO₄, 300 mM NaCl, 20 mM imidazole and 0.05% Tween-20, pH 8 was added to each well to wash for 5 min, 3 times. The chips were then rinsed with water and allowed to dry. EAM was added to spots. The chips were ready for MS analysis. Broth volume optimization is shown in FIG. 3 while a comparison of the two methods of E. coli disruption are shown in FIG. 4.

What is claimed is:

1. An apparatus for expression and capture of biomolecules comprising:
a. at least one reaction vessel defining a reaction space;
b. an expression system housed within the reaction space, wherein the expression system expresses a first biomolecule; and,
c. a solid support having an adsorbent surface that binds the first biomolecule wherein the solid support is in fluid communication with the reaction space.

2. The apparatus of claim 1, wherein the expression system comprises a cell-free system.

3. The apparatus of claim 1, wherein the expression system comprises a cell-based system.

4. The apparatus of claim 1, wherein the expression system comprises both a cell-free system and a cell-based system.

5. The apparatus of claim 1, wherein the expression system expresses at least a second biomolecule.

6. The apparatus of claim 1, wherein the first biomolecule comprises a capture moiety that binds to the adsorbent surface.

7. The apparatus of claim 1, wherein the first biomolecule comprises a detectable moiety.

8. The apparatus of claim 1, wherein the apparatus comprises a plurality of reaction vessels.

9. The apparatus of claim 1, wherein the solid support is not an integral part of the reaction vessel.

10. The apparatus of claim 9, wherein beads comprise the solid support.

11. The apparatus of claim 1, wherein the solid support comprises a hydrogel.

12. The apparatus of claim 1, wherein the adsorbent surface comprises a specific binding reagent selected from the group consisting of: an antibody, a receptor, an antigen and a ligand.

13. The apparatus of claim 1, wherein the solid support is an integral part of the reaction vessel.

14. The apparatus of claim 13, wherein the solid support is comprised within a biochip.

15. The apparatus of claim 14, wherein the apparatus comprises a plurality of reaction vessels and the biochip comprises a plurality of addressable locations comprising an adsorbent surface in fluid communication with a plurality of reaction spaces.

16. The apparatus of claim 14, wherein the biochip is an MS probe.

17. The apparatus of claim 16, wherein the MS probe is a SEAC probe.

18. The apparatus of claim 16, wherein the MS probe is a SEAC/SEND probe.

19. The apparatus of claim 15, wherein the biochip is an MS probe.

20. The apparatus of claim 19, wherein the MS probe is a SEAC probe.

21. The apparatus of claim 19, wherein the MS probe is a SEAC/SEND probe.

22. The apparatus of claim 13, wherein the reaction vessel is comprised within a multi-well microtiter plate and the solid support is comprised within a wall and/or floor of each well.

23. The apparatus of claim 22 wherein the wells of the microtiter plate comprise closed bottoms.

24. The apparatus of claim 22 wherein the microtiter plate is a filter plate.

25. A system for detecting a biomolecule comprising:
a. an apparatus comprising:
i. at least one reaction vessel defining a reaction space;
ii. an expression system housed within the reaction space wherein the expression system expresses a first biomolecule;
iii. a solid support having an adsorbent surface that binds the first biomolecule wherein the solid support is in fluid communication with the reaction space; and,
b. a detector comprising means for detecting a molecule immobilized on the adsorbent surface.

26. The system of claim 25, wherein the means for detecting comprises a mass spectrometer that detects mass-to-charge ratio of the molecule.

27. The system of claim 25, wherein the molecule is fluorescently labeled and the detector comprises a fluorimeter.

28. The system of claim 25, wherein the detector comprises means for detecting a parameter selected from the group consisting of absorbance, reflectance, transmittance, birefringence, refractive index, and diffraction.

29. The system of claim 25, wherein the means for detecting is selected from the group consisting of surface plasmon resonance, ellipsometry, resonant mirror techniques, grating coupled waveguide techniques and multipolar resonance spectroscopy.

30. The system of claim 25, wherein the expression system is a cell-based expression system and wherein the system further comprises a sonicating device.

31. A method for expressing and capturing a biomolecule, comprising:
a. providing an apparatus comprising:
i. at least one reaction vessel defining a reaction space; and
ii. a solid support having an adsorbent surface that binds a first biomolecule wherein the solid support is in fluid communication with the reaction space;
b. providing in the reaction space an expression system that expresses the first biomolecule;
c. expressing the first biomolecule within the reaction space; and,
d. capturing the first biomolecule on the adsorbent surface.

32. The method of claim 31, wherein the expression system comprises a cell-free system.

33. The method of claim 31, wherein the expression system comprises a cell-based system.

34. The method of claim 33, further comprising disrupting the cells of the expression system after expressing the first biomolecule.

35. The method of claim 30, wherein the expression system comprises both a cell-free system and a cell-based system.

36. The method of claim 31, further comprising:
e. detecting the captured first biomolecule.

37. The method of claim 31, further comprising:
e. eluting the captured first biomolecule from the adsorbent surface.

38. The method of claim 31, wherein the first biomolecule has enzymatic activity, and wherein the method further comprises:
e. contacting the captured first biomolecule with a second biomolecule; and
f. detecting evidence of enzymatic activity on the second biomolecule.

39. The method of claim 38 wherein the enzymatic activity is selected from the group consisting of kinase activity, phosphatase activity, glycosylating activity, deglycosylating activity, lipidase activity, delipidase acitivty, transcriptase activity, DNAase activity, RNAase activity and protease activity.

40. The method of claim 31, wherein the first biomolecule has enzymatic activity, and wherein the method further comprises:
e. contacting the captured first biomolecule with a plurality of second biomolecules; and
f. detecting evidence of enzymatic activity on a plurality of second biomolecules.

41. The method of claim 40 wherein the enzymatic activity is selected from the group consisting of kinase activity, phosphatase activity, glycosylating activity, deglycosylating activity, lipidase activity, delipidase acitivty, transcriptase activity, DNAase activity, RNAase activity and protease activity.

42. The method of claim 31, wherein the first biomolecule has enzymatic activity, and wherein the method further comprises:
e. contacting the captured first biomolecule with an enzymic substrate of the first biomolecule;
f. contacting the first biomolecule with a plurality of test compounds; and
g. detecting evidence of enzymatic activity on the enzymatic substrate.

43. The method of claim 42 wherein the enzymatic activity is selected from the group consisting of kinase activity, phosphatase activity, glycosylating activity, deglycosylating activity, lipidase activity, delipidase acitivty, transcriptase activity, DNAase activity, RNAase activity and protease activity.

44. The method of claim 31, further comprising:
e. contacting the captured first biomolecule with a second biomolecule; and
f. detecting evidence of binding between the first biomolecule and the second biomolecule.

45. The method of claim 44 wherein the first biomolecule is selected from the group consisting of soluble receptors, membrane bound receptors and antibodies.

46. The method of claim 44 wherein the expression system expresses the second biomolecule.

47. The method of claim 31, further comprising:
e. contacting the captured first biomolecule with a plurality of second biomolecules; and
f. detecting evidence of binding between the first biomolecule and any of the second biomolecules.

48. The method of claim 47 wherein the first biomolecule is selected from the group consisting of soluble receptors, membrane bound receptors and antibodies.

49. The method of claim 47 wherein the expression system expresses the plurality of second biomolecules.

50. The method of claim 31, further comprising:
e. contacting the captured first biomolecule with a binding partner capable of forming a comlex with the first biomolecule;
f. contacting the first biomolecule with a test agent; and
g. detecting evidence of modulation of complex formation between the first biomolecule and the binding parther.

51. The method of claim 50 wherein the first biomolecule is selected from the group consisting of soluble receptors, membrane bound receptors and antibodies.

52. The method of any of claims 32 to 51 wherein detecting comprises fluorescence detection.

53. The method of any of claims 32 to 51 wherein detecting comprises fluorescence detection.

54. The method of any of claims 32 to 51 wherein the solid support is comprised within a biochip that is an integral part of the reaction vessel and is detachable from the reaction vessel.

55. The method of claim 54, wherein the apparatus comprises a plurality of reaction vessels and the biochip comprises a plurality of addressable locations, each addressable location having an adsorbent surface in fluid communication with a different reaction space.

56. The method of claim 54, wherein the biochip is an MS probe.

57. The method of claim 31, wherein the first biomolecule comprises a capture moiety that binds to the adsorbent surface.

58. The method of claim 31, wherein the first biomolecule comprises a detectable moiety.

59. A kit for the expression and capture of biomolecules comprising:
a. an apparatus including:
i. a chamber for housing an expression system for a biomolecule; and,
ii. a solid support having an adsorbent surface that specifically binds the biomolecule wherein the solid support is in fluid communication with the chamber; and,
b. instructions for the use of the apparatus and buffer system in expressing and capturing the biomolecule.

60. The kit of claim 59, further comprising:
c. a wash solution for washing the adsorbent surface.

61. The kit of claim 59 wherein the wash solution comprises an ionic interaction modifier, a pH mofifier, a water structure modifier, a hydrophobic interaction modifier, a chaotropic reagent or an affinity interaction displacer.

62. The kit of claim 59, further comprising:
c. an expression system for expressing the biomolecule.

63. The kit of claim 59, further comprising:
c. an expression system for expressing a plurality of biomolecules.

64. The kit of claim 59, wherein the first biomolecule is an enzyme, and the kit further comprises:
c. a substrate of the enzyme.

65. The kit of claim 59, comprising a biochip that comprises the solid support and wherein the biochip is an integral part of the reaction vessel and is detachable from the reaction vessel.

66. The kit of claim 65, wherein the apparatus comprises a plurality of reaction vessels and the biochip comprises a plurality of addressable locations comprising an adsorbent surface in communication with a plurality of reaction spaces.

67. The kit of claim 65, wherein the biochip is an MS probe.

68. A method comprising:
a. providing a learning set comprising a plurality of data objects representing expression/capture experiments, wherein the experiments are classified into a plurality of different classes based on type of expression system and wherein each data object comprises data representing specific measurement of a plurality of polypeptides from each experiment captured according to the method of claim 31; and
b. training a learning algorithm with the learning set, thereby generating a classification model, wherein the classification model classifies a data object according to expression system type.
